(19) United States
(12) Patent Application Publication
(10) Pub. No.: US 2023/0133776 A1
(43) Pub. Date: May 4, 2023

(54) METHODS FOR DIAGNOSING CANCER

(71) Applicant: Queen Mary University of London, London (GB)

(72) Inventor: Muy-Teck TEH, London (GB)

(21) Appl. No.: 17/628,951

(22) PCT Filed: Jul. 22, 2020
(86) PCT No.: PCT/EP2020/070689

§ 371 Date: Jan. 21, 2022

(30) Foreign Application Priority Data
Jul. 22, 2019 (GB) 1910444.7

(51) Int. Cl. C12Q 1/6886; G01N 33/574

(52) U.S. Cl. CPC C12Q 1/6886; G01N 33/57407; C12Q 2600/158

(57) ABSTRACT

The present application relates to methods for screening for, testing for or diagnosing cancer, in particular squamous cell carcinoma such as head and neck squamous cell carcinoma. The invention uses one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16.

### CROSS-REFERENCE TO RELATED APPLICATIONS

This application is a U.S. National Phase Application filed under 35 U.S.C. § 371 claiming benefit to International Patent Application No. PCT/EP2020/070689, filed Jul. 22, 2020, which claims the benefit of priority from United Kingdom Application No. 1910444.7, filed Jul. 22, 2019, the contents of each of which are incorporated herein by reference in their entirety.

### REFERENCE TO A “SEQUENCE LISTING,” A TABLE, OR A COMPUTER PROGRAM LISTING APPENDIX SUBMITTED AS AN ASCII TEST FILE

The present application hereby incorporates by reference the entire contents of the text file named “206189-0040-00US_Updated_Sequence_Listing.txt” in ASCII format. The text file containing the Sequence Listing of the present application was created on Jun. 8, 2022, and is size 7,006 bytes in size.

The present application relates to methods for diagnosing cancer, in particular to methods for diagnosing squamous cell carcinoma.

### BACKGROUND

Despite advances in treatment options for head and neck squamous cell carcinoma (HNSCC), the 5-year survival rate has not improved over the last half century (50-60%), mainly because many malignancies are not diagnosed until late stages of the disease. Published data showed that over 70% HNSCC patients have some form of pre-existing lesions amenable to early diagnosis and risk stratification (1-5). Hence, the potential to reduce the morbidity and mortality of HNSCC through early detection is of critical importance. Oral premalignant disorders (OPMDs), 70% of which precedes HNSCC (1, 2, 6), are very common and easy to identify but clinicians are unable to differentiate between high- and low-risk OPMDs through histopathological gold standard method for cancer diagnosis, which is based on subjective opinion provided by pathologists (3, 4, 7, 8). As there is currently no quantitative method available for cancer risk assessment, the majority of OPMD patients are put on stressful, time-consuming and expensive surveillance (1-3, 5, 7). Although there are many screening adjuncts in the market, none of them to date is able to identify high-risk from benign lesions with significant confidence (1, 3-5, 7, 8). Worldwide head and neck cancer incidence ranks 1 st for India (incidence: 767,000 cases in 2012), 2nd for USA (260,000 cases/yr) and 3rd for China (213,000 cases/yr).

Oral premalignant disorders (OPMDs) are very common and some of these converts to head and neck squamous cell carcinomas (HNSCC). A systematic review on 992 OPMD patients estimated a malignancy conversion rate of 12%. Given 213,100 HNSCC cases in China each year, and 70% of HNSCCs preceded by OPMDs, the estimated total number of at risk OPMDs would therefore be over 1.24 million cases/yr. If qMIDS is able to identify 12% (149,100 cases/yr) of high-risk OPMDs, this would mean that 88% (1.1 million cases/yr) of resources on long-term surveillance could be saved and/or redirected to manage and treat the 12% high-risk patients.

Current clinicopathological features of OPMDs are not indicative of tumour aggressiveness (1, 3). Furthermore, there are no large randomised clinical trials to direct the most appropriate treatment strategy for OPMDs (9, 10). Hence, most OPMD patients are indiscriminately put on time consuming, costly and stressful surveillance (1, 3). Such “waiting game” creates unnecessary stress and anxiety in majority of low risk patients (88%), whilst delaying and under-treating minority of high-risk patients (12%) (6). A systematic review on OPMD estimated a malignancy conversion rate of 12% (6). In China alone, the estimated total number of OPMDs is approximately 788,000 cases/year given that 135,100 HNSCC cases each year (11) and 70% of HNSCC preceded by OPMDs (2). Most patients only seek clinicians when their tumours have grown to advance stages at which they are difficult to treat or untreatable. Delayed treatment directly causes poor long-term morbidity and survival (1, 3, 12, 13). The current lack of a ‘case-finding’ diagnostic test results in ineffective patient management and unnecessary long-term financial burden to both patients and healthcare establishments.

With a multigene test such as the quantitative Malignancy Index Diagnostic System (qMIDS) which requires only 1 mm³ tissues for diagnosis (14, and WO2012013931), it has been previously shown qMIDS was able to detect malignant cells in otherwise clinicopathologically “normal-looking” biopsy tissues from HNSCC patients. Unfortunately, due to aforementioned factors, OPMD patients are generally not biopsied and even if biopsied, they were small biopsy reserved for histopathology. Furthermore, OPMD study requires long-term (>5-10 years) clinical outcome data for correlation with molecular profile of the initial OPMD biopsy sample. Therefore, it has not been possible to obtain a sufficient number of OPMD tissue samples to carry out statistically viable investigations. The closest alternative and ethically permissive specimens available for research are margin and tumour core samples from HNSCC patients.

There remains in the art a need for an accurate and non-invasive test for squamous cell carcinoma that has a high sensitivity and specificity and avoids false positive and false negative results.

### SUMMARY OF THE INVENTION

The present inventors have developed a new panel of biomarkers that us useful in the detection of cancers such as squamous cell carcinoma, and specifically HNSCC, comprising up to 14 target biomarkers and 2 reference biomarkers that has improved accuracy (combination of sensitivity and specificity). The rate of false negatives and false positives is reduced compared to biomarkers and biomarker panels of the prior art. Additionally, the positive predictive value and negative predictive value of the new biomarker panel is increased compared to the biomarkers and biomarker panels of the prior art. The invention provides significant improvements over current diagnostic tests for HNSCC, which employ visual/optical techniques the are large and expensive to setup therefore not accessible to low resource populations. Although some adjuncts may be helpful (eg. Lugol's iodine dye) for guiding the best site for biopsy, they do not quantify cancer risks. Saliva/serum/exfoliated cell-based tests suffers from poor sensitivity and are unable to locate the lesion site for biopsy. Brush biopsy is a good non-invasive technique, but due to its limited material collected, it has been shown to be ineffective for ‘case finding’ (finding high risk cases). Most importantly, all non-invasive techniques ultimately require pathologists' confirmation by tissue biopsy histopathology, and therefore these adjuncts are not cost-effective. Due to the lack of confidence in current screening adjuncts and the requirement of histopathological confirmation to inform treatment decisions, a recent UK clinical audit study found that 71% of clinicians do not use any adjuncts for assessing patients with OPMD. Hence, there is an urgent need for a tool such as qMIDS which is an affordable, simple and reliable molecular tool to provide objective measures of cancer risk. The present invention could be adopted by primary care and/or outpatient settings. The tiny biopsy sampling size (1 mm, approximately half a grain of rice) renders the invention accessible to rural, resource-poor settings without needing an expensive setup, such as a dental chair required by conventional incisional biopsy for histopathology. Dentists could perform a cost-effective simple suture-free oral punch biopsy. Unlike histopathology, careful orientation of tissue specimen is not required, thereby further minimising sample handling errors. Biopsy preparation, biomarker quantification and data analysis could be automated, negating the requirement for a highly-skilled technician, further reducing staffing cost and negating sample handling error. Diagnostic results could generally be obtained within 2 hours upon receipt of sample. The accessibility of the invention to rural populations in particular and its sensitivity for early cancer detection may potentially revolutionise HNSCC diagnosis and improve survival.

In a first aspect of the invention, there is provided a method of screening for, testing for or diagnosing cancer, comprising determining the amount of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a sample obtained from a patient.

In some embodiments of the invention, the method may comprise determining the amount of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a sample obtained from a patient, comparing the amount of the determined biomarkers in the sample from the patient to the amount of the biomarkers in or of a normal control. A difference in the amount of the biomarkers in the sample from the patient compared to the amount of the biomarkers in or of the normal control is associated with the presence of cancer or is associated with a risk of developing cancer.

In a second aspect of the invention, there is provided a method for monitoring the progression of cancer in a patient, the method comprising determining the amount of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a sample obtained from a patient, and comparing the amount of one or more of the same biomarkers in a sample obtained from the same patient at a different point in time.

In some embodiments of the invention, the method may comprise (a) determining the amount of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a sample obtained from a patient, (b) comparing the amount of the determined biomarkers in the sample from the patient to the amount of the biomarkers in or of a normal control, and (c) repeating steps (a) and (b) at two or more time intervals. A change in the difference in the amount of the biomarkers in the sample from the patient compared to the amount of the biomarkers in or of the normal control over time may be associated with an change in the progression of cancer. Accordingly, the methods of the present invention can be used to detect the onset, progression, stabilisation, amelioration and/or remission of cancer.

In a third aspect of the invention, there is provided a method of treating a patient for cancer, comprising determining the amount of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a sample obtained from a patient and proceeding with treatment if cancer is diagnosed, suspected or predicted. In some aspects, the invention provides a method of treatment is performed on a patient who has been diagnosed, or suspected of having cancer, or is predicted to develop cancer at an earlier point in time using a method of the present disclosure.

In a fourth aspect of the invention, there is provided one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16, or a combination thereof, for use in screening for, testing for or diagnosing cancer.

In a fifth aspect of the invention, there is provided the use of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a method of screening for, testing for or diagnosing cancer

In a sixth aspect of the invention, there is provided a kit for testing for cancer comprising means for detecting the level of expression of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a sample obtained from a patient.

All of the embodiments of the invention may further comprise the use of one or more reference genes, for example one or both of YAP1 and POLR2A.

### BRIEF DESCRIPTION OF THE FIGURES

Reference is made to a number of Figures as follows

FIG. 1. Individual gene expression pattern in 1761 independent clinical samples (normal/margin and core HNSCC samples) in correlation with qMIDS index values (scattered dot-plots, left panel) and segregated beeswarm plots (cut-off at 4.0, right panel). Data points in grey and red indicate qMIDS <4.0 and >4.0, respectively. Regression R² and t-test P-values are listed in FIG. 2.

FIG. 2. Various statistical methods for gene selection analysis on HNSCC clinical samples. A, Distribution methods using either equal, skewed or Gaussian distribution for grouping samples based on their qMIDS values. Insets showed histograms of qMIDS groupings (6 groups). Linear and polynomial regression analyses were applied on each distribution method. Fold change were also calculated between group 1-3 and group 4-6. R² and t-test P-values were normalised and an over-all average values were obtained for each gene. Colour grading (from Red to Yellow) indicates the strength of each gene in correlation with qMIDS. B, Threshold method is based on qMIDS cut-off value at 4.0 (14). Gene expression data were either raw (relative to reference genes) or normalised (Log 2 Ratio) values. C, Final selection summary of data from A and B. Selection were made for genes with an average score of >7.

FIG. 3. Biomarker genes and their functional groups in qMIDS^{V1} and qMIDS^{V2}. Diagrams indicate the removal of less influential genes from qMIDS^{V1} and addition of new genes and functional involvement of stroma matrix and immune modulation in qMIDS^{V2}.

FIG. 4. Case study using a single HNSCC tumour core tissue biopsy for qMIDS^{V1} and qMIDS^{V2} comparison. A, Photograph showing the cut site of a strip of tissue which was subsequently cut into 10 pieces of 1 mm³ tissue fragments. Each fragment were subjected to qMIDSV1 and qMIDSV2 assay and their corresponding qMIDS indexes were shown below. B, Data from A were plotted as box-whisker dot plots (box horizontal lines represent: median and 25-75% percentiles, whiskers represent lowest and highest values, outliers are beyond the whiskers), t-test were performed. P-values were indicated in the panel above. C, Paired and unpaired margin and tumour core sample comparisons. Similar to methods in A & B, each sample were cut into 9-24 fragments for qMIDS^{V1} and qMIDS^{V2} comparison., paired (n=7 patients) and unpaired (n=10) margin and tumour core samples were analysed. Top panel shows box-whisker dot plots (box horizontal lines represent: median and 25-75% percentiles, whiskers represent lowest and highest values, outliers are beyond the whiskers) of individual samples. Panels below showed average values from each sample and statistical t-test P-values.

FIG. 5. Independent diagnostic test efficiency comparison between qMIDS^{V1} and qMIDS^{V2} on HNSCC samples. A, Box-whisker dot plots (box horizontal lines represent: median and 25-75% percentiles, whiskers represent lowest and highest values, outliers are beyond the whiskers) showing the segregation of data and t-test analysis P-values for qMIDS^{V1} and qMIDS^{V2}. B, Diagnostic test efficiency analyses for qMIDS^{V1} and qMIDS^{V2}. Statistical results are shown in panel C. TN, true negative; FN, false negative; FP, false positive; TP, true positive. D, Data from panel A were separately subjected to ROC analysis showing the comparison between qMIDS^{V1} and qMIDS^{V2}.

FIG. 6—Primer sequence table for qMIDS^{V2}biomarkers.

FIG. 7—qMIDS^{V1} vs ^{V2} 384-well assay format and protocols A, qMIDS^{V1} vs ^{V2} assay layout for 5 samples in duplicates. B, qPCR reaction composition per well. C, Master mix preparation for each sample sufficient for n=32 wells. D, Primer (Step 1) and master mix (Step 2) loading procedures, and qPCR cycling protocol (Step 3).

FIG. 8—Melting curves of each biomarker showing a single melting peak to demonstrate qPCR primer specificity.

FIG. 9—Effect of removing one of the biomarkers from the panel of 14 test biomarkers on the diagnostic performance of qMIDS^{V2}. A, a table showing the diagnostic test efficiency details of removing one biomarkers. A normalized overall efficiency scores were calculated to summarise the diagnostic efficiency for each biomarker removed. B, Graphical representation of the overall efficiency scores from panel A. C, Data in panel A were subjected to ROC analysis for comparisons.

FIG. 10—Diagnostic efficiency comparisons between qMIDS^{V2} vs qMIDS^{V2}* (minus 4 less effective biomarkers from the panel of 14 test biomarkers of qMIDS^{V2}). A, HNSCC (paired margin and tumour cores) and neck lymph-node metastatic tissue samples were measured by either qMIDS^{V2} or qMIDS^{V2}*. B, Diagnostic efficiency analyses were performed on data collected from margin and tumour samples for qMIDS^{V2} or qMIDS^{V2}* from panel A. C, Diagnostic test efficiency table comparing between qMIDS^{V2} and qMIDS^{V2}*. D, Data from panel A were separately subjected to ROC analysis showing the comparison between qMIDS^{V1} (data from FIG. 5A), qMIDS^{V2} and qMIDS^{V2}*.

FIG. 11—Multi-cohort qMIDSV2 diagnostic efficiency comparisons across geographically and ethnically distinct HNSCC cohorts. A-B, China cohort samples (fresh frozen): A, normal oral mucosa (NOM) and oral squamous cell carcinomas (OSCC) and B, normal nasopharyngeal mucosa (NPM) and nasopharyngeal SCC (NPSCC). Student's t-test P<9.9×10−6 and Mann-Whitney U-test (P<1.6×10−4) were performed due to skewed data distribution. C-E, Indian cohort samples (FFPE): C, Samples were grouped according to histopathology: NOM, Mild/Moderate Dysplasia (Dysp), Severe Dysplasia and OSCC. D, Dysplasia samples from panel C were re-grouped according to their 5-year outcome data: no progression (benign) or progressed into OSCC (malignant). Student's t-test P<0.004 and Mann-Whitney U-test (P<2×10−6) were performed due to skewed data distribution. E, Oral submucous fibrosis (OSF), OSF with dysplasia and OSF with OSCC were compared. Outliers are indicated by black outlined symbols and t-test P-values are indicated above each chart. F, Diagnostic test efficiency were compared between China and India OSCC cohort data obtain from panel A and C. F, Diagnostic test efficiency table for OSCC comparing between UK (obtained from FIG. 10A), China and India.

### DETAILED DESCRIPTION

Within this specification, the terms “comprises” and “comprising” are interpreted to mean “includes, among other things”. These terms are not intended to be construed as “consists of only”.

Within this specification, the term “about” means plus or minus 20%, more preferably plus or minus 10%, even more preferably plus or minus 5%, most preferably plus or minus 2%.

Within this specification embodiments have been described in a way which enables a clear and concise specification to be written, but it is intended and will be appreciated that embodiments may be variously combined or separated without parting from the invention.

The term “biomarker” is used throughout the art and means a distinctive biological or biologically-derived indicator of a process, event or condition. In other words, a biomarker is indicative of a certain biological state, such as the presence of cancerous tissue.

Within this specification, the term “PCR” means the polymerase chain reaction. PCR is well known method in the art. The principle of PCR is to specifically increase the amount of a target sequence from an undetectable to detectable level.

Within this specification the term “qPCR” means real time quantitative PCR. As with PCR, this is a well-known method in the art. In classical PCR, at the end of the amplification, the product can be run on a gel for detection. In qPCR, this step can be avoided since the technology combines the DNA amplification with the immediate detection of the product in a single tube. Detection methods include those based on changes in fluorescence, which are proportional to the amount of product. Fluorescence can be monitored on each PCR cycle providing an amplification plot that allows a user to follow the reaction in real time. The amount of product detected at a certain point of the run is directly related to the initial amount of target in the sample.

Within this specification, the term “multiplex qPCR” refers to a technique that allows multiple genes to be profiled in a single sample.

The term “diagnosis” encompasses identification, confirmation, and or characterisation of the presence or absence of gastrointestinal cancer, together with the developmental stage thereof, such as early stage or late stage, or benign or metastatic cancer.

Biomarker Panels

The present invention provides a biomarker panel useful in the diagnosis of cancer, the panel comprising HOXA7, CENPA, NEK2, DNMT1, INHBA, FOXM1, TOP2A, BIRC5, MMP13, CXCL8, NR3C1, IVL, CBX7, and S100A16, along with one or more optional reference genes, such as YAP1 and/or POLR2A. In particular, the present invention provides a method of diagnosing, screening or testing for cancer comprising detecting or level of expression of a gene selected from the group consisting HOXA7, CENPA, NEK2, DNMT1, INHBA, FOXM1, TOP2A, BIRC5, MMP13, CXCL8, NR3C1, IVL, CBX7 and S100A16, and optionally one or two reference genes such as YAP1 and/or POLR2A, in a biological sample.

The biomarkers HOXA7, CENPA, NEK2, DNMT1, INHBA, FOXM1, TOP2A, BIRC5, MMP13, CXCL8, NR3C1, IVL, CBX7, and S100A16 may be considered “test” biomarkers, since a change in their level of expression may be indicative of cancer. The optional additional biomarkers may be considered “reference” biomarkers. Example reference biomarkers include ACTB, GAPDH, HPRT1, YAP1 and POLR2A. Although the present inventors have used the biomarkers YAP1 and POLR2A as reference biomarkers and have noted the invention works well, it will be appreciated by a person of skill in the art that other reference biomarkers could be used.

The genes of the biomarker panel are as follows (accession numbers are the accession numbers in the National Center for Biotechnology Information (NCBI) GenBank database, available at https://www.ncbi.nlm.nih.gov/genbank/):

| Gene | Synonyms(s) | Accession No(s). |
|---|---|---|
| HOXA7 | ANTP; HOX1; HOX1A; HOX1.1 | NM_006896.4 |
| CENPA | CenH3; CENP-A | NM_001809.4 |
| | | NM_001042426.1 |
| NEK2 | NLK1; RP67; NEK2A; HsPK21; PPP1R111 | NM_002497.4 |
| | | NM_001204183.1 |
| | | NM_001204182.1 |
| DNMT1 | AIM; DNMT; MCMT; CXXC9; HSN1E; ADCADN; | NM_001130823.3 |
| | m.Hsal | NM_001379.3 |
| | | NM_001318730.1 |
| | | NM_001318731.1 |
| INHBA | EDF; FRP | NM_002192.4 |
| FOXM1 | MPP2; HFH11; HNF-3; INS-1; MPP-2; PIG29; | NM_202002.2 |
| | FKHL16; FOXM1A; FOXM1B; FOXM1C; HFH-11; | NM_021953.3 |
| | TRIDENT; MPHOSPH2 | NM_202003.2 |
| | | NM_001243088.1 |
| | | NM_001243089.1 |
| | | XM_005253676.4 |
| | | XM_011520930.3 |
| | | XM_011520931.3 |
| | | XM_011520932.1 |
| | | XM_011520933.1 |
| | | XM_011520934.3 |
| | | XM_011520935.1 |
| TOP2A | TOP2; TP2A | NM_001067.4 |
| | | XM_005257632.1 |
| BIRC5 | API4; EPR-1 | NM_001168.3 |
| | | NM_001012270.1 |
| | | NM_001012271.1 |
| MMP13 | CLG3; MDST; MANDP1; MMP-13 | NM_002427.4 |
| CXCL8 | IL8; NAF; GCP1; LECT; LUCT; NAP1; GCP-1; | NM_000584.4 |
| | LYNAP; MDNCF; MONAP; NAP-1 | |
| NR3C1 | GR; GCR; GRL; GCCR; GCRST | NM_000176.3 |
| | | NM_001018074.1 |
| | | NM_001018075.1 |
| | | NM_001018076.1 |
| | | NM_001018077.1 |
| | | NM_001020825.1 |
| | | NM_001024094.1 |
| | | NM_001204265.1 |
| | | NM_001364180.1 |
| | | NM_001364181.1 |
| | | NM_001364182.1 |
| | | NM_001364183.1 |
| | | NM_001364184.1 |
| | | NM_001364185.1 |
| | | NM_001204258.1 |
| | | XM_005268422.3 |
| | | XM_005268423.3 |
| IVL | IVL | NM_005547.3 |
| CBX7 | CBX7 | NM_175709.5 |
| | | NM_001346743.1 |
| | | NM_001346744.1 |
| | | XM_006724174.4 |
| | | XM_006724175.4 |
| | | XM_006724176.4 |
| | | XM_006724177.4 |
| | | XM_006724178.4 |
| | | XM_011530025.3 |
| S100A16 | AAG13; S100F; DT1P1A7 | NM_001317007.1 |
| | | NM_001317008.1 |
| | | NM_080388.3 |
| YAPI | YAP; YKI; COB1; YAP2; YAP65 | NM_001130145.3 |
| | | NM_006106.4 |
| | | NM_001195044.1 |
| | | NM_001195045.1 |
| | | NM_001282098.1 |
| | | NM_001282097.1 |
| | | NM_001282099.1 |
| | | NM_001282100.1 |
| | | NM_001282101.1 |
| | | XM_005271378.3 |
| | | XM_005271380.3 |
| | | XM_005271381.3 |
| | | XM_005271383.3 |
| | | XM_011542555.2 |
| | | XM_011542556.2 |
| | | XM_017017093.1 |
| POLR2A | RPB1; RPO2; POLR2; POLRA; RPBh1; RPOL2; RpllLS; | NM_000937.5 |
| | hsRPB1; hRPB220 | |

Embodiments of the invention will generally involve the use of multiple test biomarkers, rather than test biomarkers individually. The accuracy of the test increases as the number of biomarkers used increases. In most preferred embodiments, all 14 of the test biomarkers are used (i.e. the amount of all of the 14 test biomarkers is determined). However, results can still be provided when a smaller number of test biomarkers is used.

For example, in some embodiments, the amount of at least 12 of the test biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16, is used. In some embodiments, the amount of at least 13 of the test biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 is used. In most preferred embodiments, the amount of all 14 of the test biomarkers HOXA7, CENPA, NEK2, DNMT1, INHBA, FOXM1, TOP2A, BIRC5, MMP13, CXCL8, NR3C1, IVL, CBX7 and S100A16 is used.

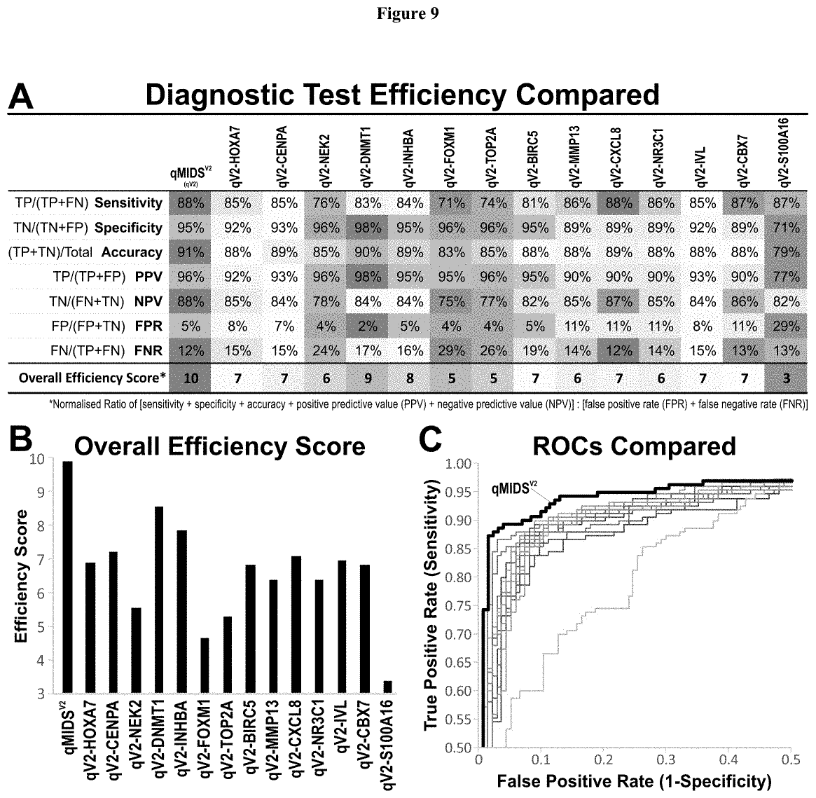

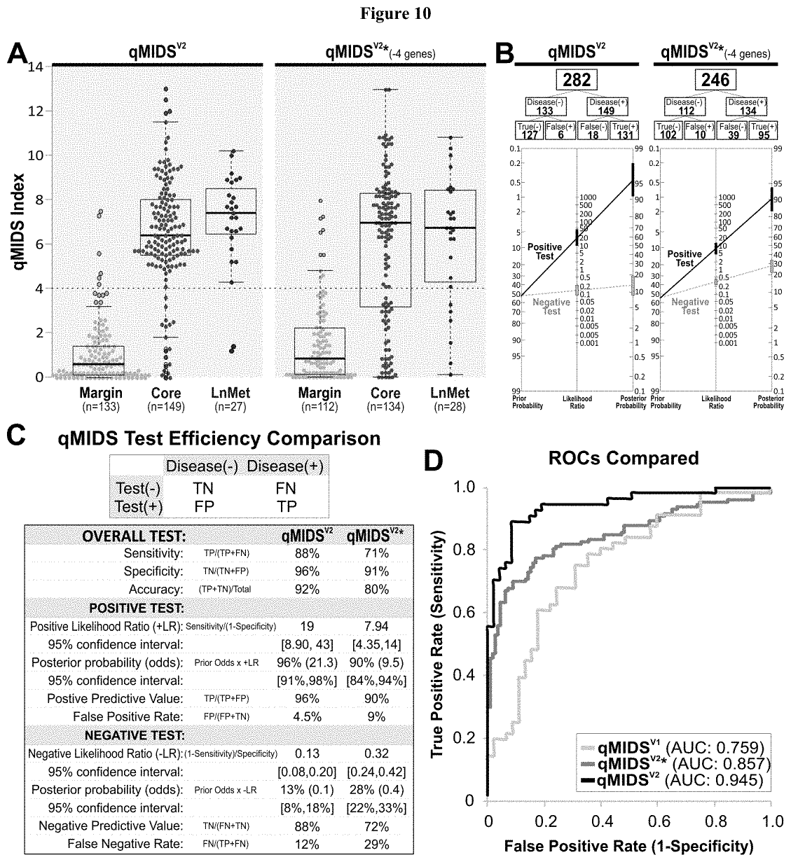

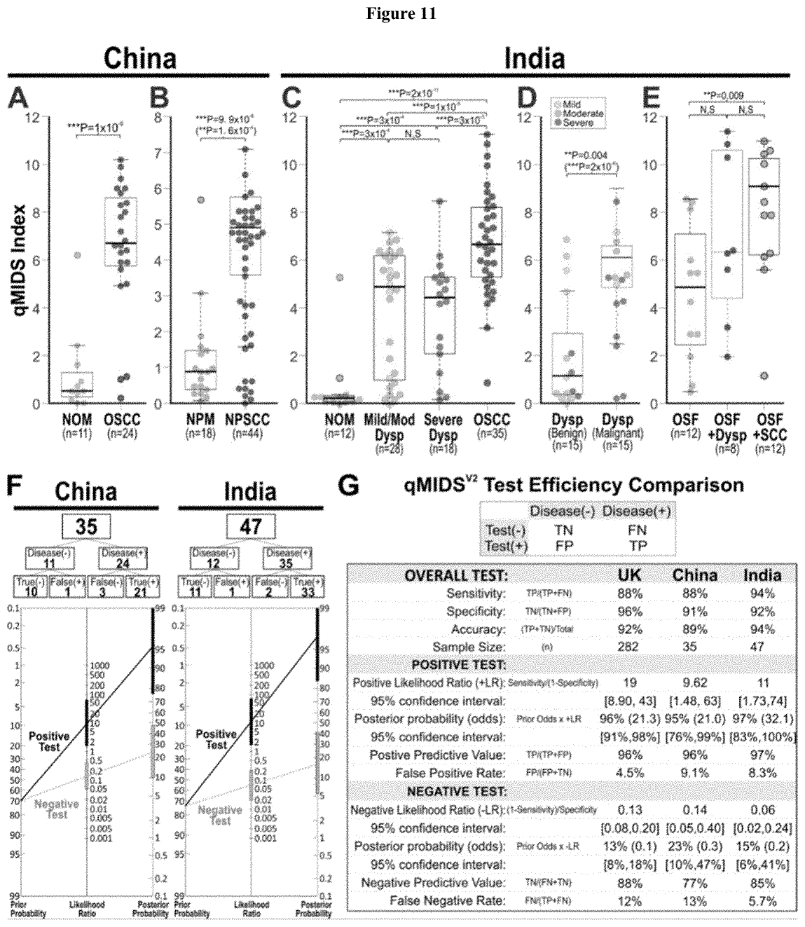

A comparison between difference biomarker panels comprising the use of 13 of the test biomarkers (i.e. the effect of removing one of each of the 14 test biomarkers) is shown in FIG. 9A. As can be seen from that figure, the use of all 14 test biomarkers provides the best results. However, a biomarker panel with one of, for example, HOXA7, CENPA, DNMT1, INHBA, BIRC5, CXCL8, IVL or CBX7 missing can still provide valuable results.

According, in some embodiments, the biomarker panel comprises:
a) all of NEK2, FOXM1, TOP2A, MMP13, NR3C1 and S100A16; and
b) at least 7 biomarkers selected from the group consisting of HOXA7, CENPA, DNMT1, INHBA, BIRC5, CXCL8, IVL and CBX7.

Such a panel provides an overall efficiency score of at least 7. The efficiency score is calculated as the ratio of [sensitivity+specificity+accuracy+positive predictive value+negative predictive value] to [false positive rate+false negative rate], and normalised as a % fraction of the sum of all the scores.

In some embodiments, the biomarker panel comprises
a) all of HOXA7, CENPA, NEK2, FOXM1, TOP2A, BIRC5, MMP13, CXCL8, NR3C1, IVL, CBX7 and S100A16; and
b) at least 1 of the biomarkers selected from the group consisting of DNMT1 and INHBA.

Such a panel provides an efficiency score of at least 8, calculated as above.

In some embodiments, the biomarker panel comprises at least all of HOXA7, CENPA, NEK2, INHBA, FOXM1, TOP2A, BIRC5, MMP13, CXCL8, NR3C1, IVL, CBX7 and S100A16. Such a panel provides an efficiency score of at least 9, calculate as above.

In some embodiments, the biomarker panel comprises all of HOXA7, CENPA, NEK2, DNMT1, INHBA, FOXM1, TOP2A, BIRC5, MMP13, CXCL8, NR3C1, IVL, CBX7 and S100A16. Such a panel provides an efficiency score of 10.

All of the biomarker panels comprising the text biomarkers can optionally be combined with one or more reference biomarkers. The reference biomarkers are those whose expression is generally stable, in particular stable across a wide variety of primary human epithelial cells, dysplastic and squamous carcinoma cell lines. The reference genes may be selected from the group consisting of ACTB, GAPDH, HPRT1, YAP1 and POLR2A. In some embodiments, the panel includes one or both of the reference genes YAP1 and POLR2A. These genes were selected as being previously validated to be among the most stable across a wide variety of primary human epithelial cells, dysplastic and squamous carcinoma cell lines (Gemenetzidis E et al., “Foxm1 upregulation is an early event in human squamous cell carcinoma and it is enhanced by nicotine during malignant transformation”, PLoS ONE 2009; 4:e4849). However, other reference genes could be used.

Depending on the biomarker and/or the cancer, it may be an upregulation or a downregulation that is indicative of cancer. The key aspect is a modulation (i.e. a change) in the level of expression or amount of one or more of the biomarkers in the sample, and in some embodiments the degree of modulation. For example, a modulation of at least about 10% or at least about 15% or at least about 20% in the level of expression or concentration of the biomarkers being tested may be indicative of cancer. The direction of the change (up or down) may depend on the biomarker being measured and/or the cancer being tested for

For example, in some embodiments, the modulation of the one or more biomarkers that may be indicative of cancer may be as follows:

| Gene | Modulation indicative of cancer |
|---|---|
| HOXA7 | Upregulation |
| CENPA | Upregulation |
| NEK2 | Upregulation |
| DNMT1 | Upregulation |
| INHBA | Upregulation |
| FOXM1 | Upregulation |
| TOP2A | Upregulation |
| BIRC5 | Upregulation |
| MMP13 | Upregulation |
| CXCL8 | Upregulation |
| NR3C1 | Upregulation |
| IVL | Downregulation |
| CBX7 | Modulation (downregulation or |
| | upregulation) |
| S100A16 | Downregulation |
| YAP1 | Reference gene |
| POLR2A | Reference gene |

In such embodiments, CBX7 expression may be downregulated or upregulated. Downregulation may be observed more frequently, although upregulation is observed in some cases, for example as observed by the present inventors in some drug resistance cancer cell lines.

Therefore, in some embodiments, cancer may be diagnosed, predicted or suspected when:
a) expression of NEK2, FOXM1, TOP2A, MMP13 and NR3C1 is upregulated
b) expression of S100A16 is downregulated; and
c) modulation of expression at least 7 biomarkers selected from the group consisting of HOXA7, CENPA, DNMT1, INHBA, BIRC5, CXCL8, IVL and CBX7 is detected, wherein modulation of expression of any of HOXA7, CENPA, DNMT1, INHBA, BIRC5, CXCL8 refers to upregulation of expression of those biomarkers, modulation of expression of IVL refers to downregulation of expression of that biomarker, and modulation of expression of CBX7 refers to downregulation or upregulation of expression of that biomarker.

In some embodiments, cancer may be diagnosed, predicted or suspected when:
a) expression of all of HOXA7, CENPA, NEK2, FOXM1, TOP2A, BIRC5, MMP13, CXCL8 and NR3C1 is upregulated;
b) expression of all of IVL, CBX7 and S100A16 is down regulated;
c) expression of CBX7 is modulated (upregulated or downregulated); and
d) at least 1 of the biomarkers selected from the group consisting of DNMT1 and INHBA is upregulated.

In some embodiments, cancer may be diagnosed, predicted or suspected when:
a) expression of all of HOXA7, CENPA, NEK2, INHBA, FOXM1, TOP2A, BIRC5, MMP13, CXCL8 and NR3C1 is upregulated;
b) expression of all of IVL and S100A16 is down regulated; and
c) expression of CBX7 is modulated (upregulated or downregulated).

In some embodiments, cancer may be diagnosed, predicted or suspected when:
a) expression of all of HOXA7, CENPA, NEK2, DNMT1, INHBA, FOXM1, TOP2A, BIRC5, MMP13, CXCL8 and NR3C1 is upregulated;
b) expression of all of IVL and S100A16 is down regulated; and
c) expression of CBX7 is modulated (upregulated or downregulated).

Modulation (upregulation or downregulation) is with respect to a control. One some embodiments, the control is from the same patient from a previous sample, to thus monitor onset or progression. Alternatively, the control may be normalised for a population, particularly a healthy or normal population, where there is no cancer. In other words, the control may consist of the level of a biomarker found in a normal control sample from a normal subject. In some embodiments, the normal control is the expression level of one or more reference genes, for example selected from YAP1 and POLR2A. The expression level of the one or more reference genes, for example YAP1 and/or POLR2A, may be from the same sample as the sample from the patient or from a different sample, for example from a patient known to have no cancer. Preferably, the expression level of one or more reference genes is from the same sample as the sample from the patient. Use of a control (also referred to as a reference) is discussed further below.

Types of Cancer

The present invention is applicable to cancers, but in particular to squamous cell carcinoma.

The methods of the invention are particularly useful in detecting early stage cancer and are more sensitive than known methods for detecting early stage cancer. Thus, the methods of the invention are particularly useful for confirming cancer when a patient has tested negative for cancer using conventional methods.

The methods described herein are applicable to various types of cancer, for example selected from oral cancer, ovarian cancer, skin cancers (including melanoma, basal cell carcinoma and squamous cell carcinoma), oesophageal cancer, lung cancer, breast cancer, kidney cancer, pancreatic cancer, prostate cancer, gastric cancer, bladder cancer, uterine cancer, colon cancer, intestinal cancer, urinary-tract cancer, blood cancer and brain cancer.

In some embodiments, the cancer is selected from metastatic carcinomas, high-grade serous ovarian adenocarcinomas, neuroblastoma, hepatocellular carcinoma, non-Hodgkin's lymphoma (including diffuse large B-cell lymphoma, follicular lymphoma, and B-cell chronic lymphocytic leukemia), colorectal carcinoma, pancreatic carcinoma, gastrointestinal stromal tumours, breast carcinomas, lymphomas, chronic myeloid leukemia and acute myeloid leukemia.

In preferred embodiments, the cancer is a squamous cell carcinoma (SCC). Squamous cell carcinomas may be selected from skin cancer, oral cancer, lung cancer, oesophageal cancer, bladder cancer, cervical cancer, prostate cancer and vaginal cancer.

In preferred embodiments, the cancer is head and neck squamous cell carcinoma (HNSCC).

In specific embodiments, the HNSCC may be oral squamous cell carcinoma (OSCC) or nasopharyngeal squamous cell carcinoma (N PSCC).

Prognosis and choice of treatment are dependent upon the stage of the cancer and the patient's general state of health. For example, in relation to oral SCC, in stage 0, abnormal cells are found in the lining of the lips and oral cavity. These abnormal cells may become cancer and spread into nearby normal tissue. Stage 0 is also called carcinoma in situ. In stage I, cancer has formed and the tumour is 2 centimetres or smaller. Cancer has not spread to the lymph nodes. In stage II, the tumour is larger than 2 centimetres but not larger than 4 centimetres, and cancer has not spread to the lymph nodes. In stage III, the tumour may be any size and has spread to a single lymph node that is 3 centimetres or smaller, on the same side of the neck as the cancer; or is larger than 4 centimetres. Stage IV is divided into stages IVA, IVB, and IVC as follows. In stage WA, the tumour has spread to nearby tissues in the lip and oral cavity; or is any size and may have spread to nearby tissues in the lip and oral cavity. Cancer has spread to 1 or more lymph nodes on one or both sides of the neck, and the involved lymph nodes are 6 centimetres or smaller. In stage IVB, the tumour may be any size and has spread to one or more lymph nodes that are larger than 6 centimetres; or has spread to the muscles or bones in the oral cavity, or to the base of the skull and/or the carotid artery. Cancer may have spread to one or more lymph nodes on one or both sides of the neck. In stage IVC, the tumour has spread beyond the lip and oral cavity to other parts of the body. The tumour may be any size and may have spread to the lymph nodes.

In relation to skin SCC, In stage 0, abnormal cells are found in the squamous cell or basal cell layer of the epidermis (topmost layer of the skin). These abnormal cells may become cancer and spread into nearby normal tissue. Stage 0 is also called carcinoma in situ. In stage I, cancer has formed and the tumour is 2 centimetres or smaller. In stage II, the tumour is larger than 2 centimetres. In stage III, cancer has spread below the skin to cartilage, muscle, or bone and/or to nearby lymph nodes, but not to other parts of the body. In stage IV, cancer has spread to other parts of the body.

It will be appreciated that the term “early stage” as used herein can be said to refer to stage 0, stage I and/or stage II, as discussed above.

With regard to the term “late stage” as used herein, it will be appreciated that this term can be said to refer to stage III and/or stage IV (for example stage IVA, IVB and/or IVC).

It will be appreciated that the “early stage” and “late stage” nature of the cancer disease states can be determined by a physician. It is also envisaged that they may be associated with non-metastatic and metastatic states, respectively.

Further provided are methods according to the present invention for monitoring a change in stage of cancer, wherein an increase in the difference in the amount of the biomarkers in the sample from the patient compared to the amount of the biomarkers in or of the normal control over time is indicative of progression of the cancer from an earlier stage to later stage of disease, for example from stage 0 to stage I, from stage Ito stage II, from stage II to stage III, from stage III to stage IV, from early stage to late stage, or from stages in between, for example from stage IVA to stage IVB or from stage IVB to stage IVC in accordance with cancer specific stages described above.

Biological Samples

The sample used for quantification of the biomarkers is a biological sample, i.e. a biological sample obtained from a patient. The biological sample may be a whole blood sample, a serum sample, a saliva sample, a cytological brush sample, or a tissue sample (biopsy), although tissue samples are particularly useful. The method may include a step of obtaining or providing the biological sample, or alternatively the sample may have already been obtained from a patient, for example in ex vivo methods.

Biological samples obtained from a patient can be stored until needed. Suitable storage methods include freezing within two hours of collection. Maintenance at −80° C. can be used for long-term storage.

The sample may be processed prior to determining the level of expression of the gene(s)/protein(s). The sample may be subject to enrichment (for example to increase the concentration of the biomarkers being quantified), centrifugation or dilution. A step of enrichment can be any suitable pre-processing method step to increase the concentration of protein in the sample. For example, the step of enrichment may comprise centrifugation and/or filtration to remove cells or unwanted analytes from the sample.

Preferably, the sample comprises biological fluid or tissue obtained from the patient. Preferably, the biological fluid or tissue comprises cellular fluid, ascites, urine, faeces, serum, pancreatic fluid, fluid obtained during endoscopy blood or saliva. In preferred embodiments, the sample comprises saliva or cells obtained from the tumour itself or surrounding cells. For example, the tissue may comprise cells from a lesion. In some embodiments, the tissue comprises cells which have been removed from the surface of a lesion. In some embodiments, the sample is obtained from a fixed, paraffin-embedded tissue.

In preferred embodiments, the sample comprises a tissue biopsy.

It is also preferred that the biological fluid is substantially or completely free of whole/intact cells. In some embodiments, the biological fluid is free of platelets and cell debris (such as that produced upon the lysis of cells). The biological fluid may be free of both prokaryotic and eukaryotic cells.

Such samples can be obtained by any number of means known in the art, such as will be apparent to the skilled person. For instance, tissue biopsy samples can be obtained using standard techniques known to a medical practitioner. Saliva samples are easily attainable, whilst blood, ascites or serum can be obtained parenterally by using a needle and syringe, for instance. Cell free or substantially cell free samples can be obtained by subjecting the sample to various techniques known to those of skill in the art which include, but are not limited to, centrifugation and filtration.

Methods of the invention may comprise a step of obtaining the sample (or samples) for a patient. In other embodiments, the methods may comprise performing the quantification of the biomarkers on a sample previously obtained from a patient.

The methods of the invention may be carried out on one test sample from a patient. Alternatively, a plurality of test samples may be taken from a patient, for example 2, 3, 4 or 5 or more samples. Each sample may be subjected to a single assay to quantify one of the biomarker panel members, or alternatively a sample may be tested for all of the biomarkers being quantified.

In some embodiments, the methods comprise at least two detection and/or quantification steps that are spaced apart temporally. The steps may be spaced apart by a few days, weeks, years or months, to determine whether the levels of the biomarkers have changed, thus indicating whether there has been a change in the progression of the cancer, enabling comparisons to be made between the level of the biomarkers in samples taken on two or more occasions, as an increase in the difference in the amount of the biomarkers in the sample from the patient compared to the amount of the biomarkers in or of the normal control over time is indicative of the onset or progression of the cancer, whereas a decrease in the difference may indicate amelioration and/or remission of the cancer.

Preferably, the difference in the level of the biomarkers is statistically significant, for example as determined by using a “t-test” providing confidence intervals of preferably at least about 80%, preferably at least about 85%, preferably at least about 90%, preferably at least about 95%, preferably at least about 99%, preferably at least about 99.5%, preferably at least about 99.95%, preferably at least about 99.99%.

Quantifying Expression of a Biomarker

Methods of the invention may comprise quantification of the one or more test and/or reference biomarkers in a sample. The amount of or a change in the level of expression may be determined in a number of ways known to the skilled person. In some embodiments, determining the amount of a biomarker in a sample may comprise quantifying the level of expression of the biomarker. This may be achieved, for example, by quantifying the amount of mRNA in the sample for a given biomarker, or quantifying the amount of protein in the sample for a given biomarker. Level of expression may also be determined by quantifying the concentration of a biomarker in a sample.

Levels of expression may be determined by, for example, quantifying the biomarkers by determining the concentration of protein in the sample. Alternatively, the amount of mRNA in the sample (such as a tissue sample) may be determined. Once the level of expression or concentration has been determined, the level can be compared to a previously measured level of expression or concentration (either in a sample from the same subject but obtained at a different point in time, or in a sample from a different subject, for example a healthy subject, i.e. a control or reference sample) to determine whether the level of expression or protein concentration is higher or lower in the sample being analysed.

Methods for detecting the levels of protein expression and methods of quantification of mRNA include any methods known in the art. For example, protein levels can be measured indirectly using DNA or mRNA arrays. Alternatively, protein levels can be measured directly by measuring the level of protein synthesis or measuring protein concentration.

DNA and mRNA arrays (microarrays), such as those provided by the present invention, comprise a series of microscopic spots of DNA or RNA oligonucleotides, each with a unique sequence of nucleotides that are able to bind complementary nucleic acid molecules. In this way the oligonucleotides are used as probes to which only the correct target sequence will hybridise under high-stringency conditions. In the present invention, the target sequence is either the coding DNA sequence or unique section thereof, corresponding to the protein whose expression is being detected, or the target sequence is the transcribed mRNA sequence, or unique section thereof, corresponding to the protein whose expression is being detected.

Directly measuring protein expression and identifying the proteins being expressed in a given sample can be done by any one of a number of methods known in the art. For example, 2-dimensional polyacrylamide gel electrophoresis (2D-PAGE) has traditionally been the tool of choice to resolve complex protein mixtures and to detect differences in protein expression patterns between normal and diseased tissue. Differentially expressed proteins observed between normal and tumour samples are separate by 2D-PAGE and detected by protein staining and differential pattern analysis. Alternatively, 2-dimensional difference gel electrophoresis (2D-DIGE) can be used, in which different protein samples are labeled with fluorescent dyes prior to 2D electrophoresis. After the electrophoresis has taken place, the gel is scanned with the excitation wavelength of each dye one after the other. This technique is particularly useful in detecting changes in protein abundance, for example when comparing a sample from a healthy subject and a sample form a diseased subject.

Commonly, proteins subjected to electrophoresis are also further characterised by mass spectrometry methods. Such mass spectrometry methods can include matrix-assisted laser desorption/ionisation time-of-flight (MALDI-TOF).

MALDI-TOF is an ionisation technique that allows the analysis of biomolecules (such as proteins, peptides and sugars), which tend to be fragile and fragment when ionised by more conventional ionisation methods. Ionisation is triggered by a laser beam (for example, a nitrogen laser) and a matrix is used to protect the biomolecule from being destroyed by direct laser beam exposure and to facilitate vaporisation and ionisation. The sample is mixed with the matrix molecule in solution and small amounts of the mixture are deposited on a surface and allowed to dry. The sample and matrix co-crystallise as the solvent evaporates.

Protein microarrays can also be used to directly detect protein expression. These are similar to DNA and mRNA microarrays in that they comprise capture molecules fixed to a solid surface. Capture molecules are most commonly antibodies specific to the proteins being detected, although antigens can be used where antibodies are being detected in serum. Further capture molecules include proteins, aptamers, nucleic acids, receptors and enzymes, which might be preferable if commercial antibodies are not available for the protein being detected. Capture molecules for use on the protein arrays can be externally synthesised, purified and attached to the array. Alternatively, they can be synthesised in-situ and be directly attached to the array. The capture molecules can be synthesised through biosynthesis, cell-free DNA expression or chemical synthesis. In-situ synthesis is possible with the latter two. There is therefore provided a protein microarray comprising capture molecules (such as antibodies) specific for each of the biomarkers being quantified immobilised on a solid support. In one embodiment of the invention, the microarray comprises capture molecules specific for each of the test biomarkers, and optionally also any reference biomarkers.

Once captured on a microarray, detection methods can be any of those known in the art. For example, fluorescence detection can be employed. It is safe, sensitive and can have a high resolution. Other detection methods include other optical methods (for example colorimetric analysis, chemiluminescence, label free Surface Plasmon Resonance analysis, microscopy, reflectance etc.), mass spectrometry, electrochemical methods (for example voltammetry and amperometry methods) and radio frequency methods (for example multipolar resonance spectroscopy).

Additional methods of determining protein concentration include mass spectrometry and/or liquid chromatography, such as LC-MS, UPLC, or a tandem UPLC-MS/MS system.

Once the level of expression or concentration has been determined, the level can be compared to a previously measured level of expression or concentration (either in a sample from the same subject but obtained at a different point in time, or in a sample from a different subject, for example a healthy subject, i.e. a control or reference sample) to determine whether the level of expression or concentration is higher or lower in the sample being analysed. The methods of the invention may further comprise a step of correlating said detection or quantification with a control or reference to determine if cancer is present, predicted or suspected, or not. Said correlation step may also detect the presence of particular types or stages of cancer and to distinguish these patients from healthy patients, in which no cancer is present, or from patients suffering from pre-cancerous conditions, such as benign lesions. Step of correlation may include comparing the amount of the measured biomarkers with the amount of the corresponding biomarkers in a reference sample, for example in a biological sample taken from a healthy patient. Generally, the method does not include the steps of determining the amount of the corresponding biomarker in a reference sample, and instead such values will have been previously determined. However, in some embodiments the methods of the invention may include carrying out the method steps from a healthy patient who is used as a control. Alternatively, the method may use reference data obtained from samples from the same patient at a previous point in time. In this way, the effectiveness of any treatment can be assessed and a prognosis for the patient determined.

Internal controls can be also used, for example quantification of one or more different biomarkers not part of the test biomarker panel. This may provide useful information regarding the relative amounts of the biomarkers in the sample, allowing the results to be adjusted for any variances according to different populations or changes introduced according to the method of sample collection, processing or storage. In some embodiments, therefore, the methods comprise quantifying the level of expression of one or more reference biomarkers (such as YAP1 and/or POLR2A).

As would be apparent to a person of skill in the art, any measurements of analyte concentration or expression may need to be normalised to take in account the type of test sample being used and/or any processing of the test sample that has occurred prior to analysis. Data normalisation also assists in identifying biologically relevant results. Invariant biomarkers may be used to determine appropriate processing of the sample. Differential expression calculations may also be conducted between different samples to determine statistical significance.

In some embodiments, detection and/or quantification of the biomarkers is by or comprises one or more of qPCR, isothermal amplification, MALDI-TOF, SELDI, via interaction with a ligand or ligands, 1-D or 2-D gel-based analysis systems, Liquid Chromatography, combined liquid chromatography and Mass spectrometry techniques including ICAT(R) or iTRAQ(R), thin-layer chromatography, NMR spectroscopy, sandwich immunoassays, enzyme linked immunosorbent assays (ELISAs), radioimmunoassays (RAI), enzyme immunoassays (EIA), lateral flow/immunochromatographic strip tests, Western Blotting, immunoprecipitation, and particle-based immunoassays including using gold, silver, or latex particles, magnetic particles or Q-dots and immunohistochemistry on tissue sections. Optionally, detection and/or quantification of the biomarkers is performed on a microtitre plate, strip format, array or on a chip.

In some embodiments, detection and/or quantification of the biomarkers is by qPCR, for example multiplex qPCR.

In some embodiments, the biomarkers are detected at the same time, for example using multiplex qPCR. In this respect, in a method which comprises detection/quantification of the test biomarkers and optionally the one or more reference biomarkers, the amount of all the genes can be measured at the same time.

Algorithms

In some embodiments, the amount of each biomarker is determined by qPCR. The difference in the amount of the biomarkers in the sample from the patient compared to the amount of the biomarkers in or of the normal control may be analysed using the algorithm:

$$MI = {\sum }_{i = 1}^{n} \vert {{Log}_{2} [ \frac{T ( {T}_{nm} )}{{T}_{m} ( {T}_{n} )} ]}_{i} \cdot {Log}_{2} [ {Q}_{4} ( {Q}_{3} - {Q}_{1} ] \qquad [ 1 \}$$

or the algorithm

$$MI = {\sum }_{i = 1}^{n} \vert {{Log}_{2} [ \frac{T ( {T}_{nm} )}{{T}_{m} ( {T}_{n} )} ]}_{i} \cdot {Log}_{2} [ {Q}_{4} ( {Q}_{2} - {Q}_{1} ] \qquad [ 2 ]$$

wherein,
MI=Malignancy Index (or the likelihood of the subject suffering from malignant cancer);

n=the number of biomarkers (also referred to as target genes herein) analysed;
T=the biomarker mRNA copy number (normalised against one or more reference genes);
Tₙ=the sum of the n biomarkers mRNA copy numbers measured;
Tₘ=the median value of T derived from a set of independently healthy normal subject samples;
Tₙₘ=the sum of the nTₘ values; and
Q1, Q2, Q3 and Q4=the first (25%), second (50%), third (75%) and fourth (100%) rank quartile of the n biomarker absolute Loge ratio distribution values for the level of each biomarker,
to provide an indication of the likelihood of the subject suffering from malignant cancer.

According to another aspect of the present invention, there is provided a method for analysing the differential expression of biomarkers between samples obtained from a patient suffering from or suspected of suffering from cancer and samples obtained from or of a normal control, the method comprising analysing the differential expression using the algorithm

$$MI = {\sum }_{i = 1}^{n} \vert {{Log}_{2} [ \frac{T ( {T}_{nm} )}{{T}_{m} ( {T}_{n} )} ]}_{i} \cdot {Log}_{2} [ {Q}_{4} ( {Q}_{3} - {Q}_{1} ] \qquad [ 1 ]$$

or the algorithm

$$MI = {\sum }_{i = 1}^{n} \vert {{Log}_{2} [ \frac{T ( {T}_{nm} )}{{T}_{m} ( {T}_{n} )} ]}_{i} \cdot {Log}_{2} [ {Q}_{4} ( {Q}_{2} - {Q}_{1} ] \qquad [ 2 ]$$

wherein,
MI=Malignancy Index (or the likelihood of the subject suffering from malignant cancer);
n=the number of biomarkers (also referred to as target genes herein) analysed;
T=the biomarker mRNA copy number (normalised against one or more reference genes);
Tₙ=the sum of the n biomarkers mRNA copy numbers measured;
Tₘ=the median value of T derived from a set of independently healthy normal subject samples;
Tₙₘ=the sum of the nTₘ values; and
Q1, Q2, Q3 and Q4=the first (25%), second (50%), third (75%) and fourth (100%) rank quartile of the n biomarker absolute Loge ratio distribution values for the level of each biomarker,

For example, in an embodiment of the present invention, wherein 14 biomarkers are analysed (for example in relation to methods for diagnosing SCC), the algorithm would be as follows:

$$MI = {\sum }_{i = 1}^{14} \vert {{Log}_{2} [ \frac{T ( {T}_{14 m} )}{{T}_{m} ( {T}_{14} )} ]}_{i} \cdot {Log}_{2} [ {Q}_{4} ( {Q}_{3} - {Q}_{1} ] \qquad [ 3 ]$$

wherein,
T represents the biomarker mRNA copy number (normalised against one or more reference genes);
T₁₄ represents the sum of the 14 biomarker mRNA copy numbers measured;
Tₘ represents a median value of T derived from a set of independent healthy primary normal subject samples;
T₁₄ₘ represents the sum of the 14Tₘ values; and
Q1, Q3 and Q4 represent the first (25%), third (75%) and fourth (100%) rank quartile of the 14 biomarker absolute Log₂ ratio distribution values for the level of each biomarker.

In some embodiments, the one or more reference genes are selected from YAP1 and POLR2A. In some embodiments, T represents the biomarker mRNA copy number normalised against two reference genes. In some embodiments, the reference genes are YAP1 and POLR2A.

In some embodiments, the amount of each biomarker is determined by qPCR and the difference in the amount of the biomarkers in the sample from the patient compared to the amount of the biomarkers in or of the normal control is analysed using the algorithm

$$MI = {\sum }_{i = 1}^{n} \vert {{Log}_{2} [ \frac{T ( {T}_{nm} )}{{T}_{m} ( {T}_{n} )} ]}_{i} \cdot {Log}_{2} [ {Q}_{4} ( {Q}_{3} - {Q}_{1} ] / R \qquad [ 1 A ]$$

or the algorithm

$$MI = {\sum }_{i = 1}^{n} \vert {{Log}_{2} [ \frac{T ( {T}_{nm} )}{{T}_{m} ( {T}_{n} )} ]}_{i} \cdot {Log}_{2} [ {Q}_{4} ( {Q}_{2} - {Q}_{1} ] / R \qquad [ 2 A ]$$

wherein,
MI=Malignancy Index (or the likelihood of the subject suffering from malignant cancer);
n=the number of biomarkers (also referred to as target genes herein) analysed;
T=the biomarker mRNA copy number (normalised against one or more reference genes);
Tₙ=the sum of the n biomarkers mRNA copy numbers measured;
Tₘ=the median value of T derived from a set of independently healthy normal subject samples;
Tₙₘ=the sum of the nTₘ values;
Q1, Q2, Q3 and Q4=the first (25%), second (50%), third (75%) and fourth (100%) rank quartile of the n biomarker absolute Log 2 ratio distribution values for the level of each biomarker; and
R=a qPCR correction factor based on R=IF((cp^{R}−26.3)<1,cp^{R}/26.3,cp^{R}−26.3), whereby cp^{R} represents the geometric mean crossing point value of the one or more reference genes measured,
to provide an indication of the likelihood of the subject suffering from malignant cancer.

According to another aspect of the present invention, there is provided a method for analysing the differential expression of biomarkers between samples obtained from a patient suffering from or suspected of suffering from cancer and samples obtained from or of a normal control, the method comprising analysing the differential expression using the algorithm

$$MI = {\sum }_{i = 1}^{n} \vert {{Log}_{2} [ \frac{T ( {T}_{nm} )}{{T}_{m} ( {T}_{n} )} ]}_{i} \cdot {Log}_{2} [ {Q}_{4} ( {Q}_{3} - {Q}_{1} ] / R \qquad [ 1 A ]$$

or the algorithm

$$MI = {\sum }_{i = 1}^{n} \vert {{Log}_{2} [ \frac{T ( {T}_{nm} )}{{T}_{m} ( {T}_{n} )} ]}_{i} \cdot {Log}_{2} [ {Q}_{4} ( {Q}_{2} - {Q}_{1} ] / R \qquad [ 2 A ]$$

wherein,
MI=Malignancy Index (or the likelihood of the subject suffering from malignant cancer);
n=the number of biomarkers (also referred to as target genes herein) analysed;
T=the biomarker mRNA copy number (normalised against one or more reference genes);
Tₙ=the sum of the n biomarkers mRNA copy numbers measured;
Tₘ=the median value of T derived from a set of independently healthy normal subject samples;
Tₙₘ=the sum of the nTₘ values;
Q1, Q2, Q3 and Q4=the first (25%), second (50%), third (75%) and fourth (100%) rank quartile of the n biomarker absolute Log 2 ratio distribution values for the level of each biomarker; and
R=a qPCR correction factor based on R=IF((cp^{R}−26.3)<1,cp^{R}/26.3,cp^{R}−26.3), whereby cp^{R} represents the geometric mean crossing point value of the one or more reference genes measured,
to provide an indication of the likelihood of the subject suffering from malignant cancer.

For example, in an embodiment of the present invention, wherein 14 biomarkers are analysed (for example in relation to methods for diagnosing SCC), the algorithm would be as follows:

$$MI = {\sum }_{i = 1}^{14} \vert {{Log}_{2} [ \frac{T ( {T}_{14 m} )}{{T}_{m} ( {T}_{14} )} ]}_{i} \cdot {Log}_{2} [ {Q}_{4} ( {Q}_{3} - {Q}_{1} ] / R \qquad [ 3 A ]$$

wherein,
T represents the biomarker mRNA copy number (normalised against one or more reference genes);

T₁₄ represents the sum of the 14 biomarker mRNA copy numbers measured;
Tₘ represents a median value of T derived from a set of independent healthy primary normal subject samples;
T₁₄ₘ represents the sum of the 14Tₘ values;
Q1, Q3 and Q4 represent the first (25%), third (75%) and fourth (100%) rank quartile of the 14 biomarker absolute Log₂ ratio distribution values for the level of each biomarker;
R represents a qPCR correction factor based on R=IF((cp^{R}−26.3)<1,cp^{R}/26.3,cp^{R}−26.3), whereby cp^{R} represents the geometric mean crossing point value of the one or more reference genes measured.

In some embodiments, the one or more reference genes are selected from YAP1 and POLR2A. In some embodiments, T represents the biomarker mRNA copy number normalised against two reference genes. In some embodiments, the reference genes are YAP1 and POLR2A.

Topological Mapping

Another aspect of the present invention relates to a method for topological mapping of a tissue sample, the method comprising:
a) dissecting a tissue sample into two or more pieces;
b) calculating a Malignancy Index (MI) value for each piece according to a method described herein; and
c) providing a malignancy heat map of the tissue sample based upon the corresponding MI values of each fragment.

In some embodiments, the tissue sample is a suspected tumour.

In some embodiments, the tissue sample is dissected into two or more pieces using a cutting grid. In some embodiments, the cutting grid comprises a plurality of cutting blades positioned to form a cutting grid. In some embodiments, the cutting grid comprises a plurality of regularly spaced intersecting blades. Optionally, the tissue sample is dissected into equal sized pieces. It will be appreciated that the number of pieces into which the tumour is dissected will depend upon the size of the tumour and the desired resolution of the resultant malignancy heat map. For example, in some embodiments, the tumour may be dissected into three or more, four or more, five or more, six or more, seven or more, eight or more, nine or more, ten or more, eleven or more, twelve or more, fifteen or more, twenty or more pieces, and so on. An advantage of the method of topological mapping is that tumour margins can be located in a given tissue sample.

### METHODS OF THE INVENTION

In general, the methods of the present invention may comprise the steps of:
a) providing a biological sample, such as a tissue sample;
b) optionally processing the sample, for example to enrich the sample for mRNA; and
c) quantification of the test biomarkers.

The methods may further comprise the step of:
d) comparison of the level of expression determined in step d) with a control or reference sample, or quantification of on more reference biomarkers; and
e) determination of a modulation in expression of the test biomarkers.

In some embodiments of the invention, the step of quantification may comprise the following steps:
a) contacting the sample with a binding partner that specifically binds to the biomarker of interest;
b) quantifying the amount of biomarker-binding partner to determine the amount of the biomarker present in the original sample.

The present invention therefore provides a reaction mixture, comprising a biological sample (such as a tissue sample, which has been optionally processed) comprises the biomarkers, wherein the biomarkers are bound to respective binding partners specific to the biomarkers. The binding partners may be, for example, oligonucleotide primers that specifically bind to mRNA or cDNA encoding the biomarkers. Alternatively, the binding partners may be, for example, antibodies that specifically bind to the biomarkers. The selective binding molecules are exogenous.

When quantifying the biomarkers using RNA, the methods may comprise a step of conducting reverse transcription to convert the mRNA encoding the biomarkers into cDNA. The methods may then further comprise a step of contact the cDNA encoding the biomarkers with one or more oligonucleotide primers that specifically bind to the cDNA encoding the biomarkers. Each biomarker may be targeted using a pair of primers (one forward and one reverse). Example suitable primers for this purpose are shown below.

| | | Forward | Reverse | |
|---|---|---|---|---|
| Gene | Loci | Primer | Primer | Bp^{a} |
| HOXA7 | 7p5-1p14 | GCCAATT | GGTAGCG | 121 |
| | | TCCGCAT | GTTGAAG | |
| | | CTACCC | TGGAAC | |
| CENPA | 21324-021 | CTGCACC | GAGAGTC | 63 |
| | | CAGTGTT | CCCGGTA | |
| | | TCTGTC | TCATCC | |
| NEK2 | 1q32.2 | CATTGGC | GAGCCAT | 90 |
| | | ACAGGCT | AGTCAAG | |
| | | CCTAC | TTCTTTC | |
| | | | CA | |
| DNMT1 | 19p13.2 | CGATGTG | TGTCCTT | 64 |
| | | GCGTCTG | GCAGGCT | |
| | | TGAG | TTACATT | |
| INHBA | 7p15-p13 | GCTCAGA | AAATTCT | 69 |
| | | CAGCTCT | CTTTCTG | |
| | | TACCACA | GTCCCCA | |
| | | | CT | |
| FOXM1 | 12p13 | ACTTTAA | CGTGCAG | 63 |
| | | GCACATT | GGAAAGG | |
| | | GCCAAGC | TTGT | |
| TOP2A | 17q21.2 | CAGTGAA | AAGCTGG | 96 |
| | | GAAGACA | ATCCCTT | |
| | | GCAGCAA | TTAGTTC | |
| | | A | C | |
| BIRC5 | 17425 | AGAACTG | ACACTGG | 104 |
| | | GCCCTTC | GCCAAGT | |
| | | TTGGA | CTGG | |
| MMP13 | 11q22.3 | TGAGCTG | AGGTAGC | 94 |
| | | GACTCAT | GCTCTGC | |
| | | TGTCGG | AAACTG | |
| CXCL8 | 4q13-q21 | AAGTTTT | TGGCATC | 74 |
| | | TGAAGAG | TTCACTG | |
| | | GGCTGAG | ATTCTTG | |
| | | A | GA | |
| NR3C1 | 5431.3 | TCCCTGG | GCTGGAT | 77 |
| | | TCGAACA | GGAGGAG | |
| | | GTTTTT | AGCTTA | |
| IVL | 1q21 | TGCCTGA | TTCCTCA | 83 |
| | | GCAAGAA | TGCTGTT | |
| | | TGTGAG | CCCAGT | |
| CBX7 | 22q13.1 | CGAGTAT | GGGGGTC | 77 |
| | | CTGGTGA | CAAGATG | |
| | | AGTGGAA | TGCT | |
| S100A1 | 1q 21 | CAAGATC | GAGCTTA | 94 |
| | | AGCAAGA | TCCGCAG | |
| | | GCAGCTT | CCTTC | |
| YAPI | 11q13 | ACAATGA | CCACTGT | 77 |
| | | CGACCAA | CTGTACT | |
| | | TAGCTCA | CTCATCT | |
| | | G | CG | |
| POLR2A | 17p13.1 | TCCGTAT | TCATCCA | 73 |
| | | TCGCATC | TCTTGTC | |
| | | ATGAAC | CACCAC | |

As noted above, the method of the invention can be carried out using an exogenous binding molecules or reagents specific for the protein or proteins being detected. “Exogenous” refers to the fact the binding molecules or reagents have been added to the sample undergoing analysis. Binding molecules and reagents are those molecules that have an affinity for the protein or proteins being detected such that they can form binding molecule/reagent-protein complexes that can be detected using any method known in the art. The binding molecule of the invention can be an antibody, an antibody fragment, a protein or an aptamer or molecularly imprinted polymeric structure. Methods of the invention may comprise contacting the biological sample with an appropriate binding molecule or molecules. Said binding molecules may form part of a kit of the invention, in particular they may form part of the biosensors of in the present invention.

Antibodies can include both monoclonal and polyclonal antibodies and can be produced by any means known in the art. Techniques for producing monoclonal and polyclonal antibodies which bind to a particular protein are now well developed in the art. They are discussed in standard immunology textbooks, for example in Roitt et al., Immunology, second edition (1989), Churchill Livingstone, London. Polyclonal antibodies can be raised by stimulating their production in a suitable animal host (e.g. a mouse, rat, guinea pig, rabbit, sheep, chicken, goat or monkey) when the antigen is injected into the animal. If necessary, an adjuvant may be administered together with the antigen. The antibodies can then be purified by virtue of their binding to antigen or as described further below. Monoclonal antibodies can be produced from hybridomas. These can be formed by fusing myeloma cells and B-lymphocyte cells which produce the desired antibody in order to form an immortal cell line. This is the well-known Kohler & Milstein technique (Kohler & Milstein (1975) Nature, 256:52-55). The antibodies may be human or humanised, or may be from other species.

After the preparation of a suitable antibody, it may be isolated or purified by one of several techniques commonly available (for example, as described in Harlow & Lane eds., Antibodies: A Laboratory Manual (1988) Cold Spring Harbor Laboratory Press). Generally, suitable techniques include peptide or protein affinity columns, high performance liquid chromatography (HPLC) or reverse phase HPLC (RP-HPLC), purification on Protein A or Protein G columns, or combinations of these techniques. Recombinant and chimeric antibodies can be prepared according to standard methods, and assayed for specificity using procedures generally available, including ELISA, ABC, dot-blot assays.

The present invention includes antibody derivatives which are capable of binding to antigen. Thus the present invention includes antibody fragments and synthetic constructs. Examples of antibody fragments and synthetic constructs are given in Dougall et al. (1994) Trends Biotechnol, 12:372-379.

Antibody fragments or derivatives, such as Fab, F(ab′)₂ or Fv may be used, as may single-chain antibodies (scAb) such as described by Huston et al. (993) Int Rev Immunol, 10:195-217, domain antibodies (dAbs), for example a single domain antibody, or antibody-like single domain antigen-binding receptors. In addition antibody fragments and immunoglobulin-like molecules, peptidomimetics or non-peptide mimetics can be designed to mimic the binding activity of antibodies. Fv fragments can be modified to produce a synthetic construct known as a single chain Fv (scFv) molecule. This includes a peptide linker covalently joining VH and VL regions which contribute to the stability of the molecule. The present invention therefore also extends to single chain antibodies or scAbs.

Other synthetic constructs include CDR peptides. These are synthetic peptides comprising antigen binding determinants. These molecules are usually conformationally restricted organic rings which mimic the structure of a CDR loop and which include antigen-interactive side chains. Synthetic constructs also include chimeric molecules. Thus, for example, humanised (or primatised) antibodies or derivatives thereof are within the scope of the present invention. An example of a humanised antibody is an antibody having human framework regions, but rodent hypervariable regions. Synthetic constructs also include molecules comprising a covalently linked moiety which provides the molecule with some desirable property in addition to antigen binding. For example the moiety may be a label (e.g. a detectable label, such as a fluorescent or radioactive label) or a pharmaceutically active agent.

In those embodiments of the invention in which the binding molecule is an antibody or antibody fragment, the method of the invention can be performed using any immunological technique known in the art. For example, ELISA, radio immunoassays or similar techniques may be utilised. In general, an appropriate autoantibody is immobilised on a solid surface and the sample to be tested is brought into contact with the autoantibody. If the cancer marker protein recognised by the autoantibody is present in the sample, an antibody-marker complex is formed. The complex can then be directed or quantitatively measured using, for example, a labeled secondary antibody which specifically recognises an epitope of the marker protein. The secondary antibody may be labeled with biochemical markers such as, for example, horseradish peroxidase (HRP) or alkaline phosphatase (AP), and detection of the complex can be achieved by the addition of a substrate for the enzyme which generates a colorimetric, chemiluminescent or fluorescent product. Alternatively, the presence of the complex may be determined by addition of a marker protein labeled with a detectable label, for example an appropriate enzyme. In this case, the amount of enzymatic activity measured is inversely proportional to the quantity of complex formed and a negative control is needed as a reference to determining the presence of antigen in the sample. Another method for detecting the complex may utilise antibodies or antigens that have been labeled with radioisotopes followed by a measure of radioactivity. Examples of radioactive labels for antigens include ³H, ¹⁴C and ¹²⁵I.

Aptamers are oligonucleotides or peptide molecules that bind a specific target molecule. Oligonucleotide aptamers include DNA aptamer and RNA aptamers. Aptamers can be created by an in vitro selection process from pools of random sequence oligonucleotides or peptides. Aptamers can be optionally combined with ribozymes to self-cleave in the presence of their target molecule.

Aptamers can be made by any process known in the art. For example, a process through which aptamers may be identified is systematic evolution of ligands by exponential enrichment (SELEX). This involves repetitively reducing the complexity of a library of molecules by partitioning on the basis of selective binding to the target molecule, followed by re-amplification. A library of potential aptamers is incubated with the target protein before the unbound members are partitioned from the bound members. The bound members are recovered and amplified (for example, by polymerase chain reaction) in order to produce a library of reduced complexity (an enriched pool). The enriched pool is used to initiate a second cycle of SELEX. The binding of subsequent enriched pools to the target protein is monitored cycle by cycle. An enriched pool is cloned once it is judged that the proportion of binding molecules has risen to an adequate level. The binding molecules are then analysed individually. SELEX is reviewed in Fitzwater & Polisky (1996) Methods Enzymol, 267:275-301.

Methods of Diagnosis

The present invention also provides a method of diagnosis for cancer comprising detecting the level of expression or concentration of one or more biomarkers in a biological sample (i.e. one or more of HOXA7, CENPA, NEK2, DNMT1, INHBA, FOXM1, TOP2A, BIRC5, MMP13, CXCL8, NR3C1, IVL, CBX7 and S100A16). The presence of cancer can be determined by detecting a change in gene expression or protein concentration as compared with the level of expression or protein concentration of the corresponding genes or proteins in samples taken from healthy control subjects.

In a further embodiment of the invention there is provided a gene selected from the group consisting of HOXA7, CENPA, NEK2, DNMT1, INHBA, FOXM1, TOP2A, BIRC5, MMP13, CXCL8, NR3C1, IVL, CBX7 and S100A16, or a combination thereof, for use in diagnosing cancer.

In a further embodiment of the invention, there is provided a combination of genes for use in diagnosing cancer, wherein the combination of genes comprises:
a) all of NEK2, FOXM1, TOP2A, MMP13, NR3C1 and S100A16; and
b) at least 7 biomarkers selected from the group consisting of HOXA7, CENPA, DNMT1, INHBA, BIRC5, CXCL8, IVL and CBX7.

In a further embodiment of the invention, there is provided a combination of genes for use in diagnosing cancer, wherein the combination of genes comprises:
a) all of HOXA7, CENPA, NEK2, FOXM1, TOP2A, BIRC5, MMP13, CXCL8, NR3C1, IVL, CBX7 and S100A16; and
b) at least 1 of the biomarkers selected from the group consisting of DNMT1 and INHBA.

In a further embodiment of the invention, there is provided a combination of genes for use in diagnosing cancer, wherein the combination of genes comprises at least all of HOXA7, CENPA, NEK2, INHBA, FOXM1, TOP2A, BIRC5, MMP13, CXCL8, NR3C1, IVL, CBX7 and S100A16.

In a further embodiment of the invention, there is provided a combination of genes for use in diagnosing cancer, wherein the combination of genes comprises HOXA7, CENPA, NEK2, DNMT1, INHBA, FOXM1, TOP2A, BIRC5, MMP13, CXCL8, NR3C1, IVL, CBX7 and S100A16.

Methods of Treatment

In another embodiment of the invention there is provided a method of treating or preventing cancer in a patient, comprising quantifying one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a biological sample obtained from a patient, and administering treatment for cancer if cancer is detected, predicted or suspected. Methods of treating cancer may include resecting the tumour and/or administering chemotherapy and/or radiotherapy to the patient. The biomarkers may be quantified by determining the level of gene expression (for example determining the mRNA concentration) or by determining the protein concentration.

In a further embodiment of the invention, there is provided a method of treating or preventing cancer in a patient, comprising quantifying a combination of biomarkers in a biological sample obtained from a patient, and administering treatment for cancer if cancer is detected, predicted or suspected, wherein the combination of biomarkers comprises:
a) all of NEK2, FOXM1, TOP2A, MMP13, NR3C1 and S100A16; and
b) at least 7 biomarkers selected from the group consisting of HOXA7, CENPA, DNMT1, INHBA, BIRC5, CXCL8, IVL and CBX7.

In a further embodiment of the invention, there is provided a method of treating or preventing cancer in a patient, comprising quantifying a combination of biomarkers in a biological sample obtained from a patient, and administering treatment for cancer if cancer is detected, predicted or suspected, wherein the combination of biomarkers comprises:
a) all of HOXA7, CENPA, NEK2, FOXM1, TOP2A, BIRC5, MMP13, CXCL8, NR3C1, IVL, CBX7 and S100A16; and
b) at least 1 of the biomarkers selected from the group consisting of DNMT1 and INHBA.

In a further embodiment of the invention, there is provided a method of treating or preventing cancer in a patient, comprising quantifying a combination of biomarkers in a biological sample obtained from a patient, and administering treatment for cancer if cancer is detected, predicted or suspected, wherein the combination of biomarkers comprises HOXA7, CENPA, NEK2, INHBA, FOXM1, TOP2A, BIRC5, MMP13, CXCL8, NR3C1, IVL, CBX7 and S100A16.

In a further embodiment of the invention, there is provided a method of treating or preventing cancer in a patient, comprising quantifying a combination of biomarkers in a biological sample obtained from a patient, and administering treatment for cancer if cancer is detected, predicted or suspected, wherein the combination of biomarkers comprises HOXA7, CENPA, NEK2, DNMT1, INHBA, FOXM1, TOP2A, BIRC5, MMP13, CXCL8, NR3C1, IVL, CBX7 and S100A16.

The methods of treating cancer of the present invention may be particularly useful in the treatment of early-stage cancer. The methods of preventing cancer are particularly useful in the prevention of late-stage cancer.

In some embodiments, the methods of treatment are performed on patients who have been identified as having a particular level of expression of the biomarkers in a biological sample. Said level of expression is one that it is indicative of cancer for each of the biomarkers that have been quantified. Accordingly, a method of treating cancer, comprising resecting any pancreatic tumour and/or administering chemotherapy and/or radiotherapy in a patient in whom cancer has been diagnosed using a method of the present invention, is provided.

In some embodiments, the methods of treatment might not include the actual step of administering the treatment. For example, the methods may instead comprise generating a report comprising the level of expression of the quantified biomarkers and/or an indication that the level of expression of the quantified biomarkers are up or down regulated compared to control. This information may then be used by a physician to determine what, if any, treatment should be applied to the patient. In some embodiments, the methods may recommend a patient receive treatment for cancer based on the results of the quantification of the biomarkers.

In a still further embodiment of the invention there is provided a method for determining the suitability of a patient for treatment for cancer, comprising detecting the level of expression of the biomarkers, or combinations thereof, in a sample, comparing the level of expression of the quantified biomarkers with one or more controls or reference biomarkers, and deciding whether or not to proceed with treatment for cancer if cancer is diagnosed or suspected.

In some embodiments of the invention, the methods may further comprise treating a patient for cancer if cancer is detected or suspected. If possible, treatment for may comprise resecting the tumour and optionally radiotherapy. Treatment may alternatively or additional involve treatment by chemotherapy and/or immunotherapy. Treatment by chemotherapy may include administration of gemcitabine and/or Folfirinox. Folfirinox is a combination of fluorouracil (5-FU), irinotecan, oxaliplatin and folinic acid (leucovorin). Treatment regimens involving Folfirinox may comprise administration of oxaliplatin, followed by folinic acid, followed by irinotecan (alternatively irinotecan may be administered at the same time as folinic acid), followed by 5-FU. Immunotherapy may comprise administration of one or more immune checkpoint inhibitors. Given the present application is useful for early detection of cancer, treatment may preferably comprise surgical removal of the tumour. The present invention could also be used as a prognostic tool to guide later state treatment strategies.

There is also provided a method of monitoring a patient's response to therapy, comprising determining the level of expression of at least one of the biomarkers of interest in a biological sample obtained from a patient that has previously received therapy for cancer (for example chemotherapy and/or radiotherapy). In some embodiments, the level of expression is compared with the level of expression for the same biomarker or biomarkers in a sample obtained from a patient before receiving the therapy. A decision can then be made on whether to continue the therapy or to try an alternative therapy based on the comparison of the levels of expression.

In one embodiment, there is therefore provided a method comprising:
a) determining the level of expression of at least one test biomarker, or combination of test biomarkers, in a biological sample obtained from a patient that has previously received therapy for cancer;
b) comparing the level of expression of the test biomarker or biomarkers determined in step a) with a previously determined level of expression of the same test biomarker or biomarkers (i.e. determined prior to the treatment for cancer); and
c) maintaining, changing or withdrawing the therapy for cancer.

The method may comprise a prior step of administering the therapy for cancer to the patient. In another embodiment, the method may also comprise a pre-step of determining the level of expression of at least one test biomarker, or combination thereof, in a biological sample obtained from the same patient prior to administration of the therapy. In step c), the therapy for cancer may be maintained if an appropriate adjustment in the level(s) of expression of the test biomarker or biomarkers is determined. For example, if there is a reduction in the expression of one or more of the biomarkers found to be up-regulated in cancer, or an increase in the expression of one or more of the biomarkers found to be down-regulated in cancer, then treatment may be maintained. If the levels of expression have altered sufficiently, for example back to what may be considered healthy or low-risk levels, then treatment for cancer may be withdrawn. If the levels of expression are unchanged or have worsened (for example there is an increase in the expression of one or more of the biomarkers found to be up-regulated in cancer, and/or there is a decrease in the expression of one or more of the biomarkers found to be down-regulated in cancer), this may be indicative of a worsening of the patient's condition, and hence an alternative therapy for cancer may be attempted. In this way, drug candidates useful in the treatment of cancer or can be screened.

In another embodiment of the invention, there is provided a method identifying a drug useful for the treatment of cancer, comprising:
a) quantifying the expression or concentration of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a biological sample obtained from a patient;
b) administering a candidate drug to the patient;
c) quantifying the expression or concentration of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a biological sample obtained from the same patient at a point in time after administration of the candidate drug; and
d) comparing the value determined in step (a) with the value determined in step (c), wherein a modulation in the level of expression of one or more of the biomarkers (for example a decrease in the level of expression or concentration of one or more of the biomarkers whose upregulation is indicative of cancer, and/or an increase in the level of expression or concentration of one or more of the biomarkers whose downregulation is indicative of cancer) between the two samples identifies the drug candidate as a possible treatment for cancer.

Kits and Biosensors

In a still further embodiment of the invention there is provided a kit of parts for testing for cancer comprising a means for quantifying the expression or concentration of HOXA7, CENPA, NEK2, DNMT1, INHBA, FOXM1, TOP2A, BIRC5, MMP13, CXCL8, NR3C1, IVL, CBX7 or S100A16, or combinations thereof. The means may be any suitable detection means.

In some embodiments, the kit may comprise means for quantifying the expression or concentration of:
a) all of NEK2, FOXM1, TOP2A, MMP13, NR3C1 and S100A16; and
b) at least 7 biomarkers selected from the group consisting of HOXA7, CENPA, DNMT1, INHBA, BIRC5, CXCL8, IVL and CBX7.

In some embodiments, the kit may comprise means for quantifying the expression or concentration of:
a) all of HOXA7, CENPA, NEK2, FOXM1, TOP2A, BIRC5, MMP13, CXCL8, NR3C1, IVL, CBX7 and S100A16; and
b) at least 1 of the biomarkers selected from the group consisting of DNMT1 and INHBA.

In some embodiments, the kit may comprise means for quantifying the expression or concentration of HOXA7, CENPA, NEK2, INHBA, FOXM1, TOP2A, BIRC5, MMP13, CXCL8, NR3C1, IVL, CBX7 and S100A16.

In some embodiments, the kit may comprise means for quantifying the expression or concentration of HOXA7, CENPA, NEK2, DNMT1, INHBA, FOXM1, TOP2A, BIRC5, MMP13, CXCL8, NR3C1, IVL, CBX7 and S100A16

The methods of the invention may comprise the use of one or more detection means for detecting the biomarkers, which may form part of the kits of the invention.

In some embodiments, the detection means comprise one or more magnetic beads conjugated to one or more biomarker-specific oligonucleotides. For example, an oligonucleotide may be provided for each of the biomarkers to be detected.

In some embodiments, the detection means may comprise one or more magnetic beads conjugated to one or more biomarker specific oligonucleotides, wherein the amount of the one or more biomarker specific oligonucleotides present in the detection means inversely correlates with the concentration of the biomarkers in or of a normal control. Optionally, the one or more magnetic beads are conjugated with poly-T.

The kit of parts of the invention may comprise a biosensor. A biosensor incorporates a biological sensing element and provides information on a biological sample, for example the presence (or absence) or concentration of an analyte. Specifically, they combine a biorecognition component (a bioreceptor) with a physiochemical detector for detection and/or quantification of an analyte (such as a protein).

The bioreceptor specifically interacts with or binds to the analyte of interest and may be, for example, an antibody or antibody fragment, an enzyme, a nucleic acid, an organelle, a cell, a biological tissue, imprinted molecule or a small molecule. The bioreceptor may be immobilised on a support, for example a metal, glass or polymer support, or a 3-dimensional lattice support, such as a hydrogel support.

Biosensors are often classified according to the type of biotransducer present. For example, the biosensor may be an electrochemical (such as a potentiometric), electronic, piezoelectric, gravimetric, pyroelectric biosensor or ion channel switch biosensor. The transducer translates the interaction between the analyte of interest and the bioreceptor into a quantifiable signal such that the amount of analyte present can be determined accurately. Optical biosensors may rely on the surface plasmon resonance resulting from the interaction between the bioreceptor and the analyte of interest. The SPR can hence be used to quantify the amount of analyte in a test sample. Other types of biosensor include evanescent wave biosensors, nanobiosensors and biological biosensors (for example enzymatic, nucleic acid (such as DNA), antibody, epigenetic, organelle, cell, tissue or microbial biosensors).

Dipsticks are another example of biosensor. The dipsticks of the invention may comprise a membrane. The dipsticks may further comprise a first section to which is bound an unlabeled antibody with specific affinity for the protein whose expression is being detected, a second section that is blocked with a non-reactive protein and a third section to which is bound the protein whose expression is being detected.

Dipstick techniques known in the art can be used to quickly and effectively carry out the method of the invention. Dipstick techniques include the following. A labeled antibody, for example labeled with formazan, having a specific affinity for the protein (antigen) being detected is dissolved in a sample of test fluid. A dipstick on which a nitrocellulose membrane is mounted is immersed in the reaction mixture. The membrane has one section on which non-labeled antibodies having a specific affinity for that antigen are bound. The second section is free of antibodies and is blocked with a non-reactive protein to prevent binding of labeled antibodies to the membrane. A third section of the dipstick is provided on which the antigen is bound. Reactions take place between the free antigen in the test fluid and the non-labeled antibody bonded to the membrane, as well as between the free antigen and the labeled antibody that was added to the sample. This results in a sandwich of non-labeled bonded antibody/antigen/labeled antibody over the first section of the membrane. A reaction also takes place between the labeled antibody and the bound antigen over the third section. No reaction takes places over the second section of the membrane.

The reaction is allowed to proceed for a fixed period of time or until completion is determined visually. Since formazan is a highly coloured dye, the reacted formazan-labeled antibody imparts colour to the third section, and if the antigen is present in the test fluid, to the first section as well. Since no reaction takes place over the second section, no colour is developed over that section. The second section thus acts as a negative control. In cases in which colour is imparted across the entire membrane, including the second section due to absorption of un-reacted formazan particles and, to a minor extent, of un-reacted formazan-labeled antibody, presence of the antigen is indicated by a difference in colour between the first and second sections of the membrane. The third section is provided as a positive control by demonstrating that the appropriate reactions are in fact taking place.

The length of time that the dipstick is immersed in the mixture is that which allows a difference in colour intensity to develop between the first and second sections of the membrane if the antigen is present. For most antibody-antigen reactions, colour development is essentially complete within 30 to 60 minutes. If desired, colour development of the dipstick can be monitored by simply removing the dipstick, visually checking the colour intensity across the first section of the membrane, and then re-immersing the dipstick if required. When no further change in colour intensity is seen, the reaction can be deemed complete.

The dipstick can be prepared by any conventional methods known in the art. For example, a nitrocellulose membrane is mounted at the lower end of the dipstick. A solution containing non-labeled primary antibody is applied over one section of the membrane to bind primary antibodies to the membrane. A solution containing a blocking agent (for example 1% serum albumin) is applied over another section of the membrane to prevent subsequent bonding of the primary protein to the membrane.

Dipsticks can be equipped for the detection of more than one protein at a time by including further sections to which are bound un-labeled antibodies with specific affinity for the further protein or proteins being detected and, optionally, a section to which is bound the protein being detected. In such cases, labeled antibodies with specific affinity for the protein being detected can be added to the sample such that their binding to the further section of the dipstick, and hence their presence in the sample, be detected. The antibodies can be labeled with the same dye or with a different dye. Suitable dyes, other than formazan, include acid dyes (for example anthraquinone or triphenylmethane), azo dyes (for example methyl orange or disperse orange 1), fluorescent dyes (for example fluorescein or rhodamine) or any other suitable dye known in the art such as coomassie blue, amido black, toluidine blue, fast green, Indian ink, silver nitrate and silver lactate. It is also apparent that the pre-labeled primary protein reactant is not limited to antibodies, but can include any protein or other molecule having specific affinity for a second protein to be detected in a sample.

The invention also provides protein microarrays (also known as protein chips) comprising capture molecules (such as antibodies) specific for each of the biomarkers being quantified, wherein the capture molecules are immobilised on a solid support. The solid support may be a slide, a membrane, a bead or microtitre plate. The slide may be a glass slide. The membrane may be a nitrocellulose membrane. The array may be a quantitative multiplex ELISA array. The microarrays are useful in the methods of the invention.

In particular, the present invention provides a combination of binding molecules, wherein each binding molecule specifically binds a different target analyte, and the combination of analytes the binding molecules specifically bind to HOXA7, CENPA, NEK2, DNMT1, INHBA, FOXM1, TOP2A, BIRC5, MMP13, CXCL8, NR3C1, IVL, CBX7 or S100A16, or combinations thereof, and optionally YAP1 and POLR2A.

The binding molecules may be present on a solid substrate, such an array or microarray. The binding molecules may all be present on the same solid substrate. Alternatively, the binding molecules may be present on different substrates. In some embodiments of the invention, the binding molecules are present in solution.

These kits may further comprise additional components, such as a buffer solution. Other components may include a probe or labeling molecule for the detection of the bound protein and so the necessary reagents (i.e. enzyme, buffer, etc) to perform the labeling; binding buffer; washing solution to remove all the unbound or non-specifically bound miRNAs. Binding of the binding molecules to the target analyte may occur under standard or experimentally determined conditions. The skilled person would appreciate what stringent conditions are required, depending on the biomarkers being measured. The stringent conditions may include a temperature high enough to reduce non-specific binding.

The protein arrays used may use fluorescence labeling to determine the presence and/or concentration of the biomarkers being analysed, although other labels can be used (affinity, photochemical or radioisotope tags). Label-free detection methods can also be used, such as surface plasma resonance (SRR), carbon nanotubes carbon nanowire sensors and microelectro-mechanical (MEMS) cantilevers. Near-IR fluorescent detection may be particularly useful for quantitative detection, in particular using nitrocellulose coated glass slides.

Quantitative protein analysis using antibody arrays may comprise signal amplification, multicolour detection, and competitive displacement techniques. Other techniques include scanning electron microscopy for the analysis of protein chips (SEMPC), which involves counting target-coated gold particles that interact specifically with ligands or proteins arrayed on a glass slide by utilizing backscattering electron detection. Accordingly, methods of the invention may comprise counting interactions between biomarker protein and their respective specific bindings molecules to achieve a quantitative analysis of the test sample. Quantitative protein detection and analysis is discussed further in, for example, Barry & Solovier, “Quantitative protein profiling using antibody arrays”, Proteomics, 2004, 4(12):3717-3726.

In some embodiments of the invention, the kit may comprise a cutting grid for dissecting a tissue sample into two or more pieces.

In some embodiments of the invention, the kit may comprise an mRNA extraction kit for analysing one or more biomarkers in the methods of the present invention.

Preferably, the kit comprises one or more detection means for detecting biomarkers as described herein. In some embodiments, the detection means comprises one or more magnetic beads, conjugated to one or more biomarker-specific oligonucleotides. For example, an oligonucleotide may be provided for each of the biomarkers to be detected.

In some embodiments, the detection means comprises one or more magnetic beads conjugated to one or more biomarker specific oligonucleotides, wherein the amount of the one or more biomarker specific oligonucleotides present in the detection means inversely correlates with the concentration of the biomarkers in or of a normal control. Optionally, the one or more magnetic beads are conjugated with poly-T.

In some embodiments, the detection means may be a microarray comprising a plurality of probes, wherein the microarray comprises probes specific for each of the biomarkers being detected and quantified. The probes may be oligonucleotides that specifically hybridise to the biomarkers being detected and quantified. Specific hybridization may occur under stringent conditions, for example a salt concentration of from about 0.01 M to about 1M sodium ion concentration (or other salt) at a pH of from about 7.0 to about 8.3 and a temperature of at least about 25° C.

In one embodiment, there is provided a kit of parts comprising a detection means for:
a) all of NEK2, FOXM1, TOP2A, MMP13, NR3C1 and S100A16; and
b) at least 7 biomarkers selected from the group consisting of HOXA7, CENPA, DNMT1, INHBA, BIRC5, CXCL8, IVL and CBX7.

In one embodiment, there is provided a kit of parts comprising a detection means for:
a) all of HOXA7, CENPA, NEK2, FOXM1, TOP2A, BIRC5, MMP13, CXCL8, NR3C1, IVL, CBX7 and S100A16; and
b) at least 1 of the biomarkers selected from the group consisting of DNMT1 and INHBA.

In one embodiment, there is provided a kit of parts comprising a detection means for all of HOXA7, CENPA, NEK2, INHBA, FOXM1, TOP2A, BIRC5, MMP13, CXCL8, NR3C1, IVL, CBX7 and S100A16.

In one embodiment, there is provided a kit of parts comprising a detection means for all of HOXA7, CENPA, NEK2, DNMT1, INHBA, FOXM1, TOP2A, BIRC5, MMP13, CXCL8, NR3C1, IVL, CBX7 and S100A16.

In one embodiment, there is provided a kit of parts comprising one or more magnetic beads conjugated to one or more biomarker-specific oligonucleotides, wherein collectively the biomarker-specific oligonucleotides are specific for:
a) all of NEK2, FOXM1, TOP2A, MMP13, NR3C1 and S100A16; and
b) at least 7 biomarkers selected from the group consisting of HOXA7, CENPA, DNMT1, INHBA, BIRC5, CXCL8, IVL and CBX7.

In one embodiment, there is provided a kit of parts comprising one or more magnetic beads conjugated to one or more biomarker-specific oligonucleotides, wherein collectively the biomarker-specific oligonucleotides are specific for:
a) all of HOXA7, CENPA, NEK2, FOXM1, TOP2A, BIRC5, MMP13, CXCL8, NR3C1, IVL, CBX7 and S100A16; and
b) at least 1 of the biomarkers selected from the group consisting of DNMT1 and INHBA.

In one embodiment, there is provided a kit of parts comprising one or more magnetic beads conjugated to one or more biomarker-specific oligonucleotides, wherein collectively the biomarker-specific oligonucleotides are specific for all of HOXA7, CENPA, NEK2, INHBA, FOXM1, TOP2A, BIRC5, MMP13, CXCL8, NR3C1, IVL, CBX7 and S100A16.

In one embodiment, there is provided a kit of parts comprising one or more magnetic beads conjugated to one or more biomarker-specific oligonucleotides, wherein collectively the biomarker-specific oligonucleotides are specific for all of HOXA7, CENPA, NEK2, DNMT1, INHBA, FOXM1, TOP2A, BIRC5, MMP13, CXCL8, NR3C1, IVL, CBX7 and S100A16.

In one embodiment, there is provided a DNA or RNA microarray, wherein the microarray comprises biomarker-specific oligonucleotides, wherein collectively the biomarker-specific oligonucleotides are specific for:
a) all of NEK2, FOXM1, TOP2A, MMP13, NR3C1 and S100A16; and
b) at least 7 biomarkers selected from the group consisting of HOXA7, CENPA, DNMT1, INHBA, BIRC5, CXCL8, IVL and CBX7.

In one embodiment, there is provided a DNA or RNA microarray, wherein the microarray comprises biomarker-specific oligonucleotides, wherein collectively the biomarker-specific oligonucleotides are specific for:
a) all of HOXA7, CENPA, NEK2, FOXM1, TOP2A, BIRC5, MMP13, CXCL8, NR3C1, IVL, CBX7 and S100A16; and
b) at least 1 of the biomarkers selected from the group consisting of DNMT1 and INHBA.

In one embodiment, there is provided a DNA or RNA microarray, wherein the microarray comprises biomarker-specific oligonucleotides, wherein collectively the biomarker-specific oligonucleotides are specific for all of HOXA7, CENPA, NEK2, INHBA, FOXM1, TOP2A, BIRC5, MMP13, CXCL8, NR3C1, IVL, CBX7 and S100A16.

In one embodiment, there is provided a DNA or RNA microarray, wherein the microarray comprises biomarker-specific oligonucleotides, wherein collectively the biomarker-specific oligonucleotides are specific for all of HOXA7, CENPA, NEK2, DNMT1, INHBA, FOXM1, TOP2A, BIRC5, MMP13, CXCL8, NR3C1, IVL, CBX7 and S100A16.

Also provided are kits comprising microfluidic chips for detection and quantification of the biomarkers in the biomarker panels of the invention.

In some embodiments, the kits of the invention may comprise a software program, or a computer readable medium on which a software program is stored. The software program may comprise instructions to carry out an analysis method, for example an analysis method for conducting a diagnostic method of the invention. The software program may comprise instructions for determining the level of expression or for quantifying each of the test biomarkers of interest in a sample. Alternatively, the software program may be capable of receiving information on the level of expression or the amount of each of the test biomarkers of interest in a sample. The software program may also comprise instructions to determine the presence or absence of a change in the level of expression or amount of each of the test biomarkers in the sample, for example a change compared to a control or a predetermined value. The level of expression or the amount of the biomarkers may be normalised, for example normalised with respect to one or more reference biomarkers. The software program may also comprise instructions for determining the level of expression or quantifying each of the one or more reference biomarkers in the sample, or the software program may be capable of receiving information on the level of expression or quantification of each of each of the one or more reference biomarkers in a sample

The software program may also comprise instructions for the generation of a diagnostic report, for example a diagnostic report identifying whether or not cancer is detected or suspected (or whether cancer is not detected or suspected) based on the level of expression or quantification of each of the test biomarkers of interest.

In some embodiments, the kit contains instructions for use in one or more methods of the invention.

Features for the second and subsequent aspects of the invention are as for the first aspect of the invention mutatis mutandis.

The present invention shall now be further described with reference to the following examples, which are present for the purposes of illustration only and are not to be construed as being limiting on the invention.

### EXAMPLES

Despite advances in treatment options for HNSCC, the 5-year survival rate has not improved over the last half century (50-60%), mainly because many malignancies are not diagnosed until late stages of the disease. Published data showed that over 70% HNSCC patients have some form of pre-existing lesions amenable to early diagnosis and risk stratification (1-5). Hence, the potential to reduce the morbidity and mortality of HNSCC through early detection is of critical importance. Oral premalignant disorders (OPMDs), 70% of which precedes HNSCC (1, 2, 6), are very common and easy to identify but clinicians are unable to differentiate between high- and low-risk OPMDs through histopathological gold standard method for cancer diagnosis which is based on subjective opinion provided by pathologists (3, 4, 7, 8). As there is currently no quantitative method available for cancer risk assessment, the majority of OPMD patients are put on stressful, time-consuming and expensive surveillance (1-3, 5, 7). Although there are many screening adjuncts in the market, none of them to date is able to identify high-risk from benign lesions with significant confidence (1, 35, 7, 8).

Current clinicopathological features of OPMDs are not indicative of tumour aggressiveness (1, 3). Furthermore, there are no large randomised clinical trials to direct the most appropriate treatment strategy for OPMDs (9, 10). Hence, most OPMD patients are indiscriminately put on time consuming, costly and stressful surveillance (1, 3). Such “waiting game” creates unnecessary stress and anxiety in majority of low risk patients (88%), whilst delaying and under-treating minority of high risk patients (12%) (6). A systematic review on OPMD estimated a malignancy conversion rate of 12% (6). In China alone, the estimated total number of OPMDs is approximately 788,000 cases/year given that 135,100 HNSCC cases each year (11) and 70% of HNSCC preceded by OPMDs (2). Most patients only seek clinicians when their tumours have grown to advance stages at which they are difficult to treat or untreatable. Delayed treatment directly causes poor long-term morbidity and survival (1, 3, 12, 13). The current lack of a ‘case-finding’ diagnostic test results in ineffective patient management and unnecessary long-term financial burden to both patients and healthcare establishments.

With a multigene test such as the quantitative Malignancy Index Diagnostic System (qMIDS) which requires only 1 mm3 tissues for diagnosis (14), we have previously shown promising results that qMIDS was able to detect malignant cells in otherwise clinicopathologically “normal-looking” biopsy tissues from HNSCC patients. Unfortunately, due to aforementioned factors, OPMD patients are generally not biopsied and even if biopsied, they were small biopsy reserved for histopathology. Furthermore, OPMD study requires long-term (>5-10 years) clinical outcome data for correlation with molecular profile of the initial OPMD biopsy sample. Therefore, we were unable to obtain sufficient number of OPMD tissue samples to carry out statistically viable investigations. The closest alternative and ethically permissive specimens available for research are margin and tumour core samples from HNSCC patients. Although OPMD may exhibit different molecular signature to that found in tumour, it is generally accepted that high risk OPMDs adopts a malignant signature profile during malignant conversion (2). Therefore, it is not unreasonable to use tumour signature profile as a tool for detecting early malignant conversion in OPMDs.

Over the course of development and validation of the qMIDS test for early HNSCC diagnosis and prognosis (14, 15), we have since tested over 1760 individual 1 mm3 tissue specimens donated by over 400 patients (represented by Caucasians, South Asians and East Asians). As the qMIDS test involves measuring 16 genes (14 target+2 reference) in each sample, this amounted to a large resource of gene expression data (>24,000 data points). Although all 14 target genes were originally found to be differentially expressed between normal and cancer cell lines (14), from our clinical dataset, we have shown in this study that some of these genes turned out to be less differentially expressed in biopsy samples compared to cell lines. We further demonstrated the ability to evolve and improve our qMIDS test by replacement and addition of new genes with functions in stroma/matrix and immune regulation for significantly more precise quantification of tumour biopsies.

Materials and Methods

Clinical Samples

The use of human tissue was approved by the relevant Research Ethics Committees at each institution [UK NREC: 06/MRE03/69 and Norway REK Vest: 2010/481-7 as reported previously (14). Formalin-fixed paraffin-embedded (FFPE) tissues were approved by Institutional Ethics Committee of Kasturba Hospital, Manipal, India (IEC 343/2017). All tissue samples were previously collected according to local ethical committee-approved protocols and informed patient consent was obtained from all participants (14). Clinico-histopathological reports of the tissue samples were obtained from collaborating clinicians at each institution. For the UK cohort, fresh biopsy tissues were preserved in RNALater (#AM7022, Ambion, Applied Biosystems, Warrington, UK) and stored short-term at 4° C. (1-7 days) prior to transportation and subsequent storage at −20° C. until mRNA extraction (Dynabeads mRNA Direct kit, Invitrogen). For the Norwegian cohort, frozen archival biopsy tissues (embedded in OCT medium) and tissue cryosections (50 μm thick) were preserved in RNALater prior to mRNA extraction. All frozen samples were digested with nuclease-free proteinase K at 60° C. prior to mRNA extraction. The Indian cohort of FFPE samples were each (2-8 curls of 5 μm thick sections) deparaffinised with xylene (1 mL, 1 min at 60° C. incubation, repeat once) followed by rehydration (1 mL, 100%, 90% then 70% ethanol, with each step incubate for 1 min at 60° C.) prior to air dry (60° C., 5 min) and total RNA purification (Qiagen FFPE RNeasy Kit, #73504). All samples were pseudo-anonymised and tested blindly to ensure that the qMIDS assays were performed objectively.

The qMIDS Assay

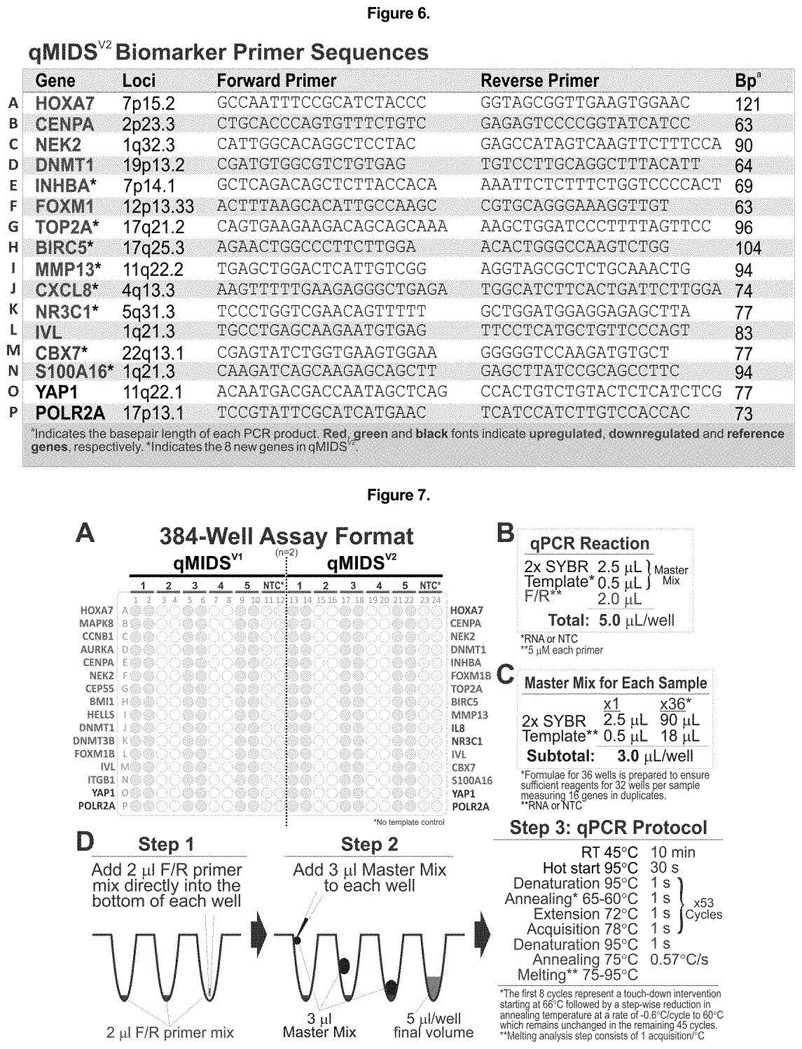

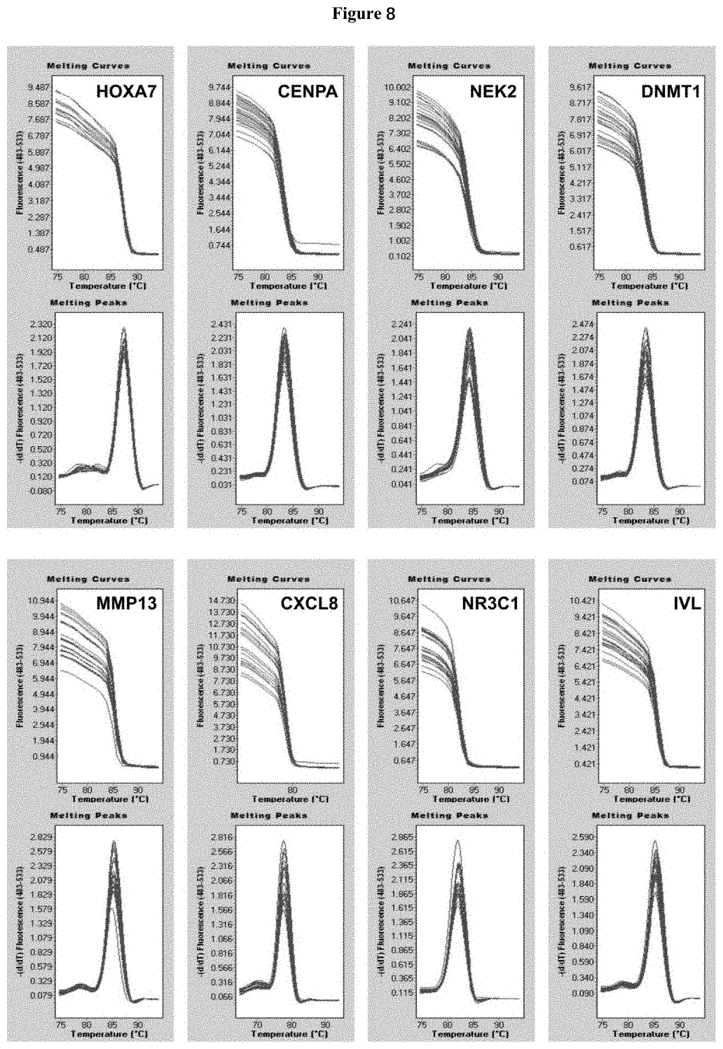

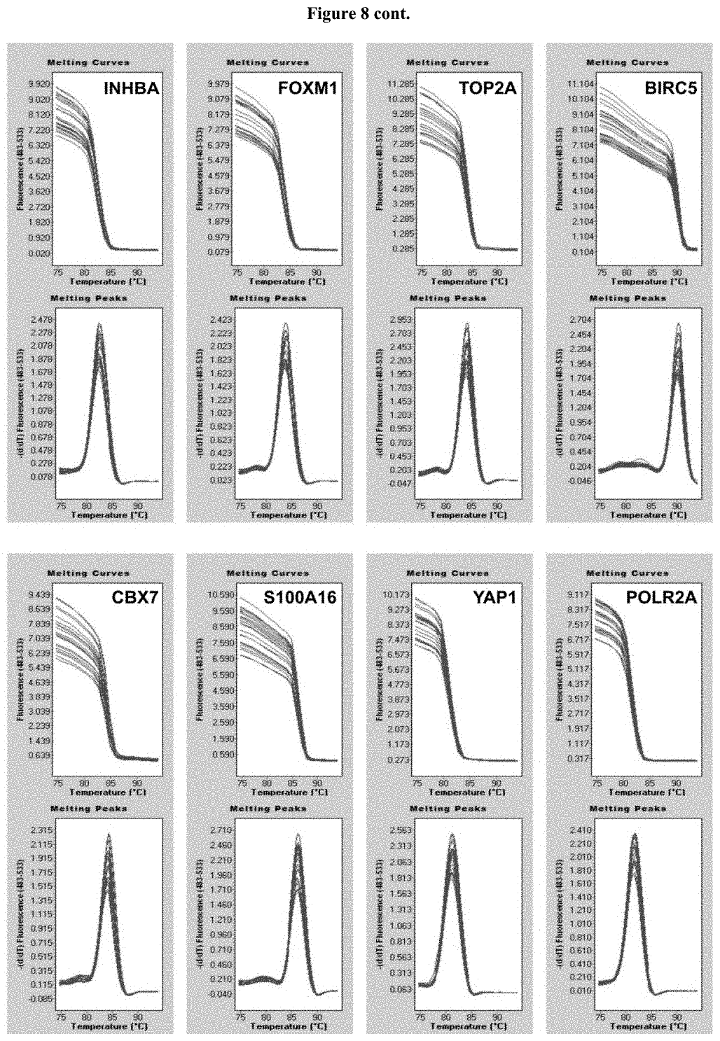

The qMIDS assay methodology was performed as described previously (14, 15). Briefly, to simplify, expedite and economise the qMIDS assay, the present assay format involves using qPCRBIO SyGrene 1-Step Go (PCRBIO, PB25.31-12) for relative quantification of 14 target genes and 2 reference genes in the LightCycler 480 qPCR system (Roche) based on our previously published protocols (14, 16-18) which are MIQE compliant (19). Briefly, thermocycling begins with 45° C. for 10 mins (for reverse transcription) followed by 95° C. for 30 s prior to 45 cycles of amplification at 95° C. for 1 s, 60° C. for 1 s, 72° C. for 1 s, 78° C. for 1 s (data acquisition). A ‘touch-down’ annealing temperature intervention (66° C. starting temperature with a step-wise reduction of 0.6° C./cycle; 8 cycles) was introduced prior to the amplification step to maximise primer specificity. Melting analysis (95° C. for 30 s, 75° C. for 30 s, 75-99° C. at a ramp rate of 0.57° C./s) was performed at the end of qPCR amplification to validate single product amplification in each well (See Supplemental FIG. 7). Relative quantification of mRNA transcripts was calculated based on an objective method using the second derivative maximum algorithm (20) (Roche). All qPCR primers and metadata of the original qMIDS (=qMIDS^{V1} were published previously (14), whereas, qMIDS^{V2} primers are provided in Supplementary Table ST1. All target genes were normalised to two stable reference genes validated previously (16) to be amongst the most stable reference genes across a wide variety of primary human epithelial cells, dysplastic and squamous carcinoma cell lines, using the GeNorm algorithm (21). The qMIDS^{V1} vs qMIDS^{V2} workflow and detail 384-well assay format setups are provided in Supplementary FIG. 7. Relative expression data were then exported into Microsoft Excel for computing qMIDS scores based on its original qMIDS algorithm (14). No template controls (NTC) were prepared by omitting tissue sample during RNA purification and eluates were used as NTCs for qMIDS assay.

Statistical Analysis

Scattered plots were analysed using polynomial regression (y=a+b1x+b2x²+b3x³) on both raw and Log 2 ratio data of each target gene to survey its correlation with qMIDS values. Statistical t-tests P values were used for differential analysis between two groups of data. Diagnostic test efficiency comparison data were calculated using a Diagnostic Test Calculator freeware (22). The qMIDS diagnostic assay efficiency tests were performed according to the STARD Initiative recommended protocol (23). Beeswarm Boxplots were created in R (version 2.13.1; The R Foundation for Statistical Computing) (24).

Results

Gene Selection

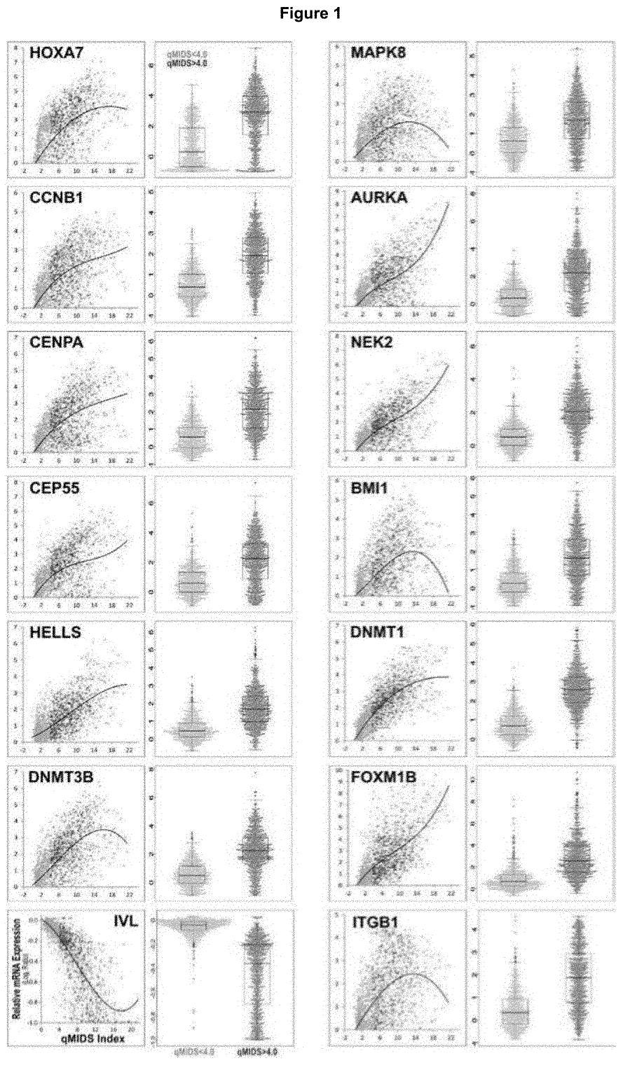

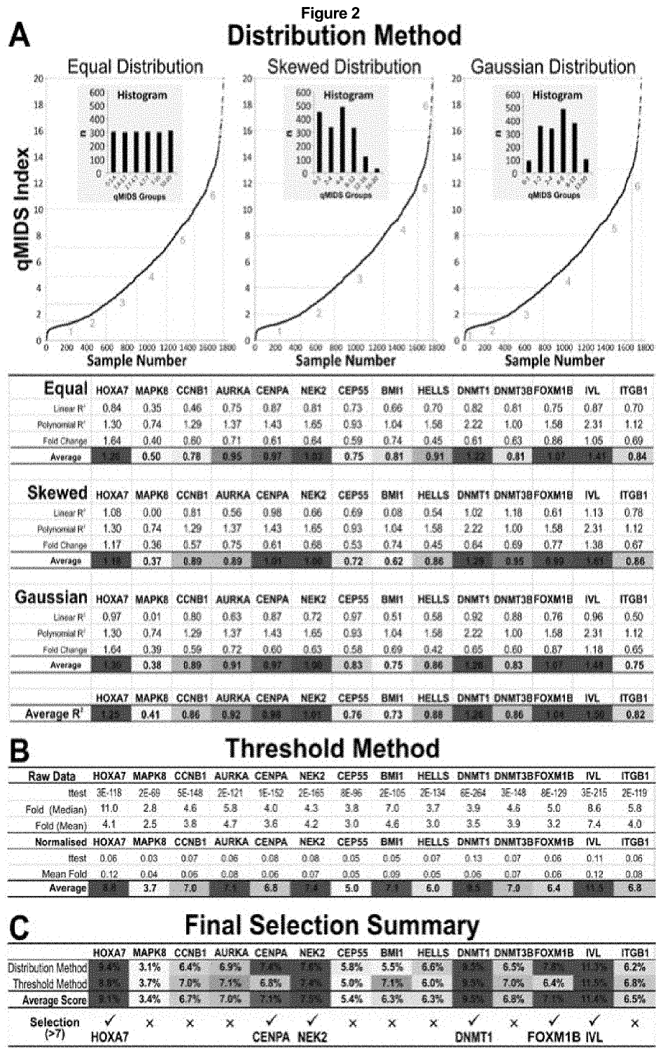

Since our first publication validating the use of qMIDS for early HNSCC diagnosis (14), we have accumulated large number (n=1761) of qMIDS data (with individual gene expression value of 14 target genes) from normal and disease tissue samples collectively donated by patients from UK and Norway, totaling to about 24,654 gene expression data points. Over the course of developing qMIDS assay for HNSCC cancer diagnosis, we noticed that some target genes were less contributory which may confound the qMIDS test efficiency. Hence, using our previous qMIDS data generated from clinical samples as a training dataset, we aimed to remove less influential genes from qMIDS. We subjected our data to two methods of analyses: 1. Distribution with correlation regression analysis, and, 2. Threshold (cut-of at 4.0) methods. For distribution method, we first performed a correlation regression analysis between each gene with qMIDS index value for each of the n=1761 samples, generating scattered dot-plots with regression analysis (FIG. 1, scattered dot-plots on left panels). We then subject our dataset to three methods of sub-groupings (following equal, skewed or Gaussian distributions) prior to linear and polynomial curve-fitting methods to access how well each gene correlated with qMIDS values (FIG. 2A). For the threshold method, we segregated samples into normal (n=1189) vs disease (n=572) based on previously determined cut-off value of 4.0 (14). Student t-test was performed on each of the 14 target genes (FIG. 1, bee-swamp plots on right panels). All correlation efficiency (R²) and t-test P values are shown in FIGS. 2A and 2C. A final average gene score were calculated from both methods and genes were selected based on an arbitrary score of >7 (FIG. 2C) whereby 6 genes (HOXA7, CENPA, NEK2, DNMT1, FOXM1, IVL) were shortlisted.

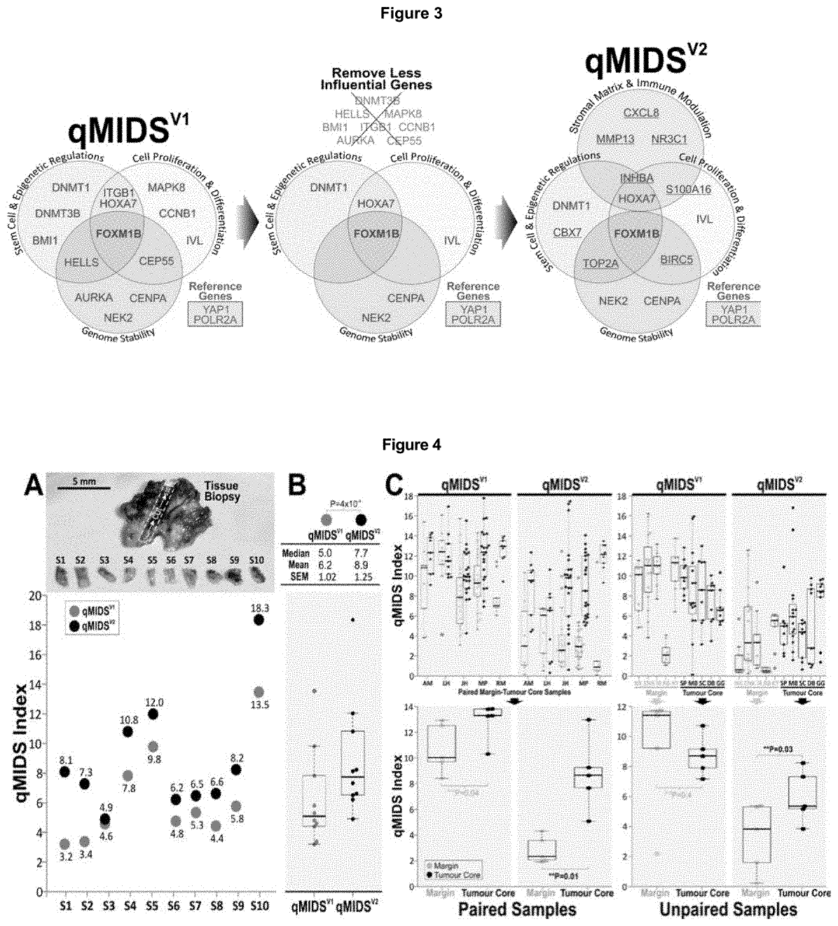

In an attempt to reduce the number of biomarkers measured in qMIDS test, we tested if a panel of 12, 10, 8 or 6 (instead of 14) genes could maintain the qMIDS diagnostic accuracy and sensitivity. Unfortunately, reducing from 14 to 12, 10, 8 or 6 genes gradually rendered the qMIDS test results unreliable (data not shown). To maintain consistency with our previously validated qMIDS assay format (14, 15) (see Supplemental FIG. 7), instead, we opted for replacing those less influential genes by adding back 8 new candidate genes (through literature and Oncomine™/GEO database searches) with functional implications in stromal matrix and immune modulation in squamous cell carcinomas (FIG. 3). A new panel of candidate genes (^{˜}20) were first shortlisted and individually tested for their significance of differentiating normal from cancer samples (data not shown). Eight most significant genes (INHBA, TOP2A, BIRC5, MMP13, CXCL8, NR3C1, CBX7, 5100A16) were then recruited into qMIDS^{V2} (FIG. 3 and Supplemental FIG. 7).

Comparison Between qMIDS^{V1} and qMIDS^{V2}

We hypothesised that by removing less influential genes and replacing with new genes involved in stroma/matrix and immune modulation will render the qMIDS test more accurate and sensitive for detecting HNSCC. To confirm this hypothesis, we compared qMIDS^{V1} vs qMIDS^{V2} on a series of clinical samples. Due to heterogeneity of tumour tissue samples, we first perform a case study on one T3 HNSCC tumour core samples. We cut this tissue specimen to obtain 10 pieces of 1 mm³ fragments (FIG. 4A). cDNA was generated from each tissue fragment and the same cDNA sample were subjected to qMIDS^{V1} and qMIDS^{V2} measurements simultaneously using 384-well format (shown in Supplementary FIG. 7). For this tumour sample, qMIDS^{V1} appeared to generate lower index values in most of the tissue fragments compared to qMIDS^{V2}. Collectively, the median/mean values for qMIDS^{V1} vs qMIDS^{V2} were 5.0/6.2 vs 7.7/8.9 (FIG. 4B) which were statistically different (P<0.0001). This indicates that qMIDS^{V2} may be more sensitive than qMIDS^{V1}. According to the clinicopathological data of this case was a T3 tumour. Therefore, a qMIDS index value of 7.7-8.9 would be more appropriate than 5-6.2, given that normal-disease cut-off value were 4.0 (14).

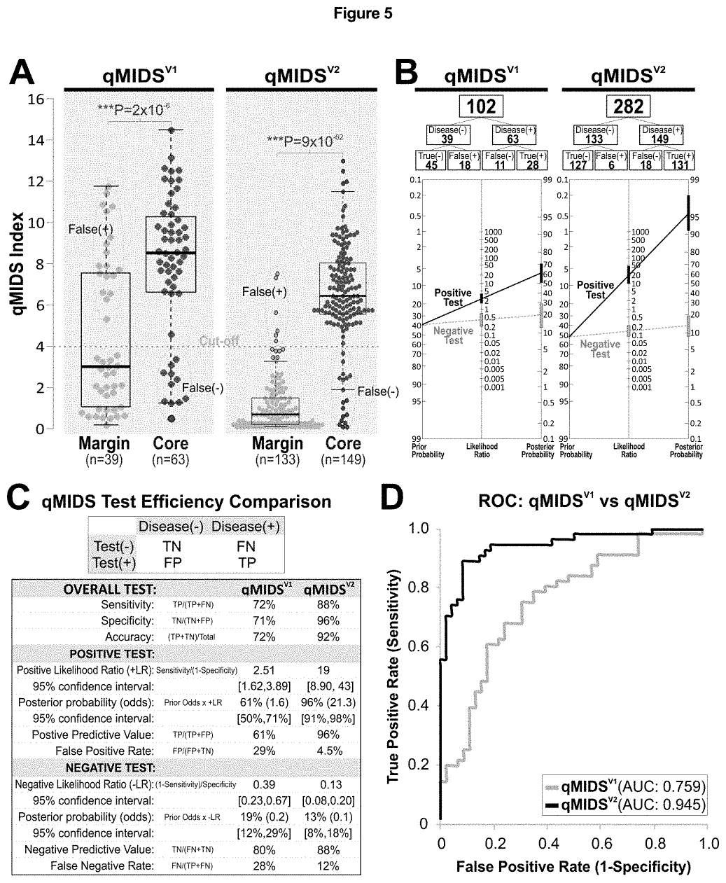

To test if qMIDS^{V2} have superior segregation power between margin and tumour core over qMIDS^{V1}, we have chosen two cohorts of patients which were previously tested and failed to be segregated by qMIDS^{V1}. The first cohort contains paired margin-tumour core samples from the same patients (n=7), the second cohort consisted of independent margin (n=5) and tumour core (n=5) samples from different patients. We have previously shown that measuring multiple sub-fragments from a single biopsy increases the diagnostic accuracy due to the ability to map out tumour heterogeneity (14). Hence, each tissue sample was cut into 9 to 24 pieces (depending on the size of biopsy) of about 1 mm³ each sub-fragment. A total of n=498 sub-fragments (from paired samples of 7 patients) and n=204 sub-fragments (unpaired samples of 10 patients) were independently analysed for qMIDS^{V1} vs qMIDS^{V2} test comparison on each fragment (FIGS. 5A and 5B). As per our original findings, our current data showed that qMIDS^{V1} failed to differentiate between margin and core tumour samples (FIG. 5C) but qMIDS^{V2} significantly segregated the samples (FIG. 5D). We concluded that for both cohorts of paired and unpaired samples, qMIDS^{V2} out performed qMIDS^{V1} in segregating margin from core tissue samples. Of particular interest, we noted that one patient (AA) showed inversed index values in both qMIDS^{V1} and qMIDS^{V2}, whereby, margin had higher index values than its tumour core (FIG. 5A). We reasoned that the two samples may have been mislabeled (reversed) during collection. Despite the inclusion of this sample, qMIDS^{V2} gave statistically significant segregation (P=0.03). If the patient AA's margin and core indexes were reversed, the segregation would then become highly significant (P=0.001).

In order to validate the diagnostic efficiency of qMIDS^{V1} compared to qMIDS^{V2}, we further tested n=102 HNSCC patient samples (FIG. 6). In agreement with above case studies (FIGS. 4 and 5), we found that qMIDS^{V2} assay indeed showed overall superior diagnostic efficiency compared to qMIDS^{V1}. Most notable were increase in sensitivity/accuracy from 71-72% in qMIDS^{V1} to 88-91% in qMIDS^{V2} (FIG. 6C). Importantly, false negative rate was reduced from 28% in qMIDS^{V1} to 9% in qMIDS^{V2}. These data confirmed that our strategy of removing less influential genes based on large gene expression datasets (>24,000 data points) from clinical tissue samples and by including genetic signatures of the tumour microenvironment (stroma/matrix/immune regulations) in additional to genetic signature of tumour cells, could significantly improve qMIDS diagnostic efficiency to enable highly precise quantitative diagnosis of HNSCC.

Discussion

In 2013, we created and validated the first multi-gene quantitative cancer diagnostic test (qMIDS) for HNSCC based on bioinformatics, cell culture and molecular selection techniques to identify key oncogenic driver genes (14). The qMIDS test was first validated on UK and Norwegian tissue samples (14) and subsequently validated in China using ethnic Han Chinese specimens (15), whereby collectively a total of over 427 specimens from Caucasians and Asians have been tested and published. Collectively, we have since amassed >1760 qMIDS data, each with 14 gene expression data points. Over the course of our continuous qMIDS development and study, we noticed that in some patients' samples, qMIDS assay were not able to differentiate between tumour core and margin samples whereby qMIDS data were discordance with histopathological reports. We suspected that some of the genes within the 14-gene panel of qMIDS were less differentially expressed in HNSCC clinical samples than were originally found in HNSCC cell lines. This is not surprising as the original panel of genes were selected based on cell line models (14).

In the attempt to fix this issue, we therefore aimed to improve the qMIDS diagnostic efficiency by exploiting our large HNSCC clinical sample gene expression data to identify and remove less influential genes from the qMIDS assay. Unfortunately, reducing genes from qMIDS led to poorer diagnostic efficiency due to assay instability. In the attempt to preserve the original qMIDS assay format (14 target genes and 2 reference genes), we therefore resorted to replacing less influential genes with new target genes. As tumour tissues contain not only tumour cells but a mix of matrix, blood vessels, infiltration of immune cells, it would be logical to involve a molecular signature that represents all these different components to obtain a more accurate picture of a tumour tissue.

Using our HNSCC clinical sample gene expression databank, we employed various statistical methods in the attempt to identify less contributory genes. We have found that of the 14 target genes, 6 genes (FOXM1, HOXA7, DNMT1, CENPA, NEK2 and IVL) showed strong and robust correlation with HNSCC malignancy whilst the remaining 8 genes were less differentially expressed. This led to the removal of 8 genes (MAPK8, CCNB1, AURKA, CEP55, BMI1, HELLS, DNMT3B and ITGB1). To preserve our previously validated qMIDS assay format, replacement with 8 new target genes selected using a combination of bioinformatics on differential gene expression databases (Oncomine/GEO), PubMed literature search and cell line screening methods as published previously (14). Amongst the 8 new genes, 5 of them (MMP13 (25, 26), INHBA (27, 28), NR3C1 (29), S100A16 (30) and CXCL8/IL8 (31-33)) are known markers involved in stroma/matrix and immune modulation of HNSCC. The remaining 3 genes filled the gaps of tumour cell regulation (CBX7 (34), TOP2A (35) and BIRC5 (36)) in stem cell, epigenetic, genomic instability, proliferation and differentiation (see FIG. 3). With the new combination of genes in qMIDS^{V2}, not surprisingly, we have demonstrated and validated on a cohort of n=102 HNSCC samples that qMIDS^{V2} assay gave overall significantly better diagnostic efficiency (21-26% increase) over qMIDS^{V1}. Importantly, the false positive rate was lowered from 29% to 14% and false negative rate was lowered from 28% to 9%.

It has been estimated in the US that early detection and treatment of HNSCC will save $100,000/patient (37) and significantly reduce the burden on the economy and society due to disability following cancer treatment (38). In the UK, it has been estimated that the total costs over a 3-year period for the management of the stages of HNSCC with cost of: precancer £1869; stage I £4914; stage II £8535; stage III £11,883 and stage IV £13,513. This study models total cost to the UK's National Health System but does not take into account any patient-related expenses or impact on productivity. The indication being that early detection of HNSCC is advantageous in purely monetary terms due to the cheaper treatment required for smaller lesions (39). Given that up to 15% of the general population may suffer from oral lesions, but the vast majority (>88%) are usually benign (40), a method is needed to identify the remaining 3-12% (1, 4, 6, 9, 40) of high risk patients whilst releasing >88% of low risk patients from time consuming, stressful and costly long-term surveillance. There is currently no consensus on whether a biopsy is taken or not from patients with OPMD. As histopathology is not accurate for predicting the risk in OPMDs, only severe cases of OPMD were biopsied whilst other OPMDs were missed. Given the sensitivity and accuracy of the qMIDS assay, we envisage that this may be a useful quantitative tool to help pathologists identify high risk OPMD lesions and release majority of low risk patients. Instead of performing a single scalpel biopsy (5-10 mm) which is highly invasive, less invasive 1 mm³ curette biopsy could be employed to minimise harm and/or enable multiple biopsies to be taken when presented with large field change in the oral compartment. The use of tissue biopsy is arguably more accurate than using saliva or brush biopsy when it comes to measuring gene expression signature identified from tumours samples. Alternative, qMIDS assay could be used as an adjunct to assist histopathological findings.

Collectively, these results demonstrated the importance of including gene signatures from the tumour microenvironment which could significantly improve tumour diagnosis, thereby lowering the chances of under or over treatments in HNSCC patients. This study also demonstrated a multi-gene diagnostic test system that is flexible and amenable to continuous evolution which allows fine-tuning improvements without compromising on overall test validity.

There is currently no diagnostic test for quantifying head & neck cancer aggressiveness. Given that both qMIDS and qMIDS-V2 are based on a universal cancer gene FOXM1 (recent Nature Medicine paper shows that it is a key gene for 39 different cancer types, Gentles et al., Nat Med, 2015), there is a potential that it could be a “universal” cancer test. We have tested qMIDS on head and neck cancer, vulva and skin cancers (data published in 2013). It was later independently validated in China (published in 2016). qMIDS-V2 is an improvement over qMIDS for better sensitivity and specificity.

### REFERENCES

1. Thomson P J, McCaul J A, Ridout F, Hutchison I L. To treat . . . Or not to treat? Clinicians' views on the management of oral potentially malignant disorders. Br J Oral Maxillofac Surg 2015; 53:1027-31.
2. Jin L I, Lamster I B, Greenspan J S, Pitts N B, Scully C, Warnakulasuriya S. Global burden of oral diseases: Emerging concepts, management and interplay with systemic health. Oral Dis 2016; 22:609-19.
3. Epstein J B, Huber M A. The benefit and risk of screening for oral potentially malignant epithelial lesions and squamous cell carcinoma. Oral Surg Oral Med Oral Pathol Oral Radiol 2015; 120:537-40.
4. Scully C. Challenges in predicting which oral mucosal potentially malignant disease will progress to neoplasia. Oral Dis 2014; 20:1-5.
5. Mehrotra R, Gupta D K. Exciting new advances in oral cancer diagnosis: Avenues to early detection. Head & neck oncology 2011; 3:33.
6. Mehanna H M, Rattay T, Smith J, McConkey C C. Treatment and follow-up of oral dysplasia—a systematic review and meta-analysis. Head Neck 2009; 31:1600-9.
7. Lingen M W, Kalmar J R, Karrison T, Speight P M. Critical evaluation of diagnostic aids for the detection of oral cancer. Oral Oncol 2008; 44:10-22.
8. Scully C, Bagan J V, Hopper C, Epstein J B. Oral cancer: Current and future diagnostic techniques. Am J Dent 2008; 21:199-209.
9. Holmstrup P, Dabelsteen E. Oral leukoplakia-to treat or not to treat. Oral Dis 2016; 22:494-7.
10. Lodi G, Franchini R, Warnakulasuriya S, Varoni E M, Sardella A, Kerr A R, et al. Interventions for treating oral leukoplakia to prevent oral cancer. Cochrane database of systematic reviews (Online) 2016; 7:CD001829.
11. Zhang S K, Zheng R, Chen Q, Zhang S, Sun X, Chen W. Oral cancer incidence and mortality in china, 2011. Chin J Cancer Res 2015; 27:44-51.
12. Haddad R I, Shin D M. Recent advances in head and neck cancer. N Engl J Med 2008; 359:1143-54.
13. Leemans C R, Braakhuis B J, Brakenhoff R H. The molecular biology of head and neck cancer. Nat Rev Cancer 2011; 11:9-22.
14. Teh M T, Hutchison I L, Costea D E, Neppelberg E, Liavaag P G, Purdie K, et al. Exploiting foxm1-orchestrated molecular network for early squamous cell carcinoma diagnosis and prognosis. Int J Cancer 2013; 132:2095-106.
15. Ma H, Dai H, Duan X, Tang Z, Liu R, Sun K, et al. Independent evaluation of a foxm1-based quantitative malignancy diagnostic system (qmids) on head and neck squamous cell carcinomas. Oncotarget 2016; 7:54555-63.
16. Gemenetzidis E, Bose A, Riaz A M, Chaplin T, Young B D, Ali M, et al. Foxm1 upregulation is an early event in human squamous cell carcinoma and it is enhanced by nicotine during malignant transformation. PLoS ONE 2009; 4:e4849.
17. Teh M T, Gemenetzidis E, Chaplin T, Young B D, Philpott M P. Upregulation of foxml induces genomic instability in human epidermal keratinocytes. Mol Cancer 2010; 9:45.
18. Waseem A, Ali M, Odell E W, Fortune F, Teh M T. Downstream targets of foxml: Cep55 and hells are cancer progression markers of head and neck squamous cell carcinoma. Oral Oncol 2010; 46:536-42.
19. Bustin S A, Benes V, Garson J A, Hellemans J, Huggett J, Kubista M, et al. The miqe guidelines: Minimum information for publication of quantitative real-time per experiments. Clin Chem 2009; 55:611-22.
20. Zhao S, Fernald R D. Comprehensive algorithm for quantitative real-time polymerase chain reaction. J Comput Biol 2005; 12:1047-64.
21. Vandesompele J, De Preter K, Pattyn F, Poppe B, Van Roy N, De Paepe A, Speleman F. Accurate normalization of real-time quantitative rt-per data by geometric averaging of multiple internal control genes. Genome Biol 2002; 3:RESEARCH0034.
22. Schwartz A, Millam G, Investigators U L. A web-based library consult service for evidence-based medicine: Technical development. BMC Med Inform Decis Mak 2006; 6:16.
23. Bossuyt P M, Reitsma J B, Standards for Reporting of Diagnostic A. The stard initiative. Lancet 2003; 361:71.
24. Juul N, Szallasi Z, Eklund A C, Li Q, Burrell R A, Gerlinger M, et al. Assessment of an rna interference screen-derived mitotic and ceramide pathway metagene as a predictor of response to neoadjuvant paclitaxel for primary triple-negative breast cancer: A retrospective analysis of five clinical trials. Lancet Oncol 2010; 11:358-65.
25. Johansson N, Airola K, Grenman R, Kariniemi AL, Saarialho-Kere U, Kahari V M. Expression of collagenase-3 (matrix metalloproteinase-13) in squamous cell carcinomas of the head and neck. Am J Pathol 1997; 151:499-
26. Stokes A, Joutsa J, Ala-Aho R, Pitchers M, Pennington C J, Martin C, et al. Expression profiles and clinical correlations of degradome components in the tumor microenvironment of head and neck squamous cell carcinoma. Clin Cancer Res 2010; 16:2022-35.
27. Khammanivong A, Sorenson B S, Ross K F, Dickerson E B, Hasina R, Lingen M W, Herzberg M C. Involvement of calprotectin (s100a8/a9) in molecular pathways associated with hnscc. Oncotarget 2016; 7:14029-47.
28. Chang W M, Lin Y F, Su C Y, Peng H Y, Chang Y C, Lai T C, et al. Dysregulation of runx2/activin-a axis upon mir-376c downregulation promotes lymph node metastasis in head and neck squamous cell carcinoma. Cancer Res 2016; 76:7140-50.
29. Long M D, Campbell M J. Pan-cancer analyses of the nuclear receptor superfamily. Nucl Receptor Res 2015; 2.
30. Sapkota D, Bruland O, Parajuli H, Osman T A, Teh M T, Johannessen A C, Costea D E. S100a16 promotes differentiation and contributes to a less aggressive tumor phenotype in oral squamous cell carcinoma. BMC Cancer 2015; 15:631.
31. Fujita Y, Okamoto M, Goda H, Tano T, Nakashiro K, Sugita A, et al. Prognostic significance of interleukin-8 and cd163-positive cell-infiltration in tumor tissues in patients with oral squamous cell carcinoma. PLoS ONE 2014; 9:e110378.
32. Li Y, St John M A, Zhou X, Kim Y, Sinha U, Jordan R C, et al. Salivary transcriptome diagnostics for oral cancer detection. Clin Cancer Res 2004; 10:8442-50.
33. Christofakis E P, Miyazaki H, Rubink D S, Yeudall W A. Roles of cxc18 in squamous cell carcinoma proliferation and migration. Oral Oncol 2008; 44:920-6.
34. Wang W, Lim W K, Leong H S, Chong F T, Lim T K, Tan D S, et al. An eleven gene molecular signature for extra-capsular spread in oral squamous cell carcinoma serves as a prognosticator of outcome in patients without nodal metastases. Oral Oncol 2015; 51:355-62.
35. Jenson E G, Baker M, Paydarfar J A, Gosselin B J, Li Z, Black C C. Mcm2/top2a (proexc) immunohistochemistry as a predictive marker in head and neck mucosal biopsies. Pathol Res Pract 2014; 210:346-50.
36. Farnebo L, Tiefenbock K, Ansell A, Thunell L K, Garvin S, Roberg K. Strong expression of survivin is associated with positive response to radiotherapy and improved overall survival in head and neck squamous cell carcinoma patients. Int J Cancer 2013; 133:1994-2003.
37. Short P F, Moran J R, Punekar R. Medical expenditures of adult cancer survivors aged <65 years in the united states. Cancer 2011; 117:2791-800.
38. Taylor J C, Terrell J E, Ronis D L, Fowler K E, Bishop C, Lambert M T, et al. Disability in patients with head and neck cancer. Arch Otolaryngol Head Neck Surg 2004; 130:764-9.
39. Speight P M, Palmer S, Moles D R, Downer M C, Smith D H, Henriksson M, Augustovski F. The cost-effectiveness of screening for oral cancer in primary care. Health Technol Assess 2006; 10:1-144, iii-iv.
40. Thomson a Oral precancer: Diagnosis and management of potentially malignant disorders. Chichester, West Sussex, UK; Hoboken, N.J.: Wiley-Blackwell, 2012.

1. A method of testing for, screening for or diagnosing cancer, comprising determining the level of expression of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a sample obtained from a patient.

2. The method of claim 1, comprising determining the level of expression of:
a) all of NEK2, FOXM1, TOP2A, MMP13, NR3C1, and S100A16; and
at least 7 biomarkers selected from the group consisting of HOXA7, CENPA, DNMT1, INHBA, BIRC5, CXCL8, IVL, AND CBX7; or
b) all of HOXA7, CENPA, NEK2, FOXM1, TOP2A, BIRC5, MMP13, CXCL8, IVL, CBX7, AND S100A16; and
at least 1 of the biomarkers selected from the group consisting of DNMT1 and INHBA; or
c) HOXA7, CENPA, NEK2, INHBA, FOXM1, TOP2A, BIRC5, MMP13, CXCL8, NR3C1, IVL, CBX7, and S100A16; or
d) HOXA7, CENPA, NEK2, DNMT1, INHBA, FOXM1, TOP2A, BIRC5, MMP13, CXCL8, NR3C1, IVL, CBX7, AND S100A16.

3-5. (canceled)

6. The method of claim 1, comprising determining the level of expression of one or more reference biomarkers, optionally wherein the one or more reference biomarkers are selected form the group consisting of YAP1, POLR2A, ACTB, GAPDH, and HPRT1.

7. (canceled)

8. The method of claim 1 wherein determining the level of expression of one or more biomarkers comprises determining the amount of mRNA or protein corresponding to each of the biomarkers in the sample.

9. The method of claim 1, wherein the cancer is squamous cell carcinoma (SCC), optionally wherein the SCC is head and neck SCC (HNSCC).

10. The method of claim 1, wherein the sample is a tissue sample.

11. The method of claim, wherein the method further comprises comparing the level of expression of the one or more biomarkers to one or more control biomarkers, optionally wherein the level of expression of the one or more control biomarkers is represented by the level of expression of one or more biomarkers selected from the group consisting of YAP1, POLR2A, ACTB, GAPDH, AND HPRTQ, further optionally wherein the level of expression of the one or more control biomarkers is the level of expression of the corresponding biomarkers from a sample obtained from a healthy patient.

12-13. (canceled)

14. The method of claim 1 wherein upregulation of any of HOXA7, CENPA, NEK2, DNMT1, INHBA, FOXM1, TOP2A, BIRC5, MMP13, CXCL8 and/or NR3C1, downregulation of any of IVL and/or S100A16, and/or modulation of CBX7 is indicative or predictive of cancer.

15. The method of claim 1, wherein the step of determining the level of expression of the one or more biomarkers comprises the use of a binding molecule or binding molecules specific for the biomarker or biomarkers whose level of expression is being determined, optionally wherein the binding molecule or binding molecules are oligonucleotides or antibodies.

16. (canceled)

17. The method of claim 1, wherein the sample is from a human.

18. The method of claim 1, wherein the sample is from a patient having or suspected of having cancer.

19-23. (canceled)

24. A kit for testing for cancer, comprising a means for quantifying the expression or concentration of one or more biomarkers selected from the group consisting of HOXA7, CENPA, NEK2, DNMT1, INHBA, FOXM1, TOP2A, BIRC5, MMP13, CXCL8, NR3C1, IVL, CBX7 and S100A16 in a sample obtained from a patient.

25. The kit of claim 24, comprising a means for quantifying the expression or concentration of:
a) all of NEK2, FOXM1, TOP2A, MMP13, NR3C1 and S100A16; and
b) at least 7 biomarkers selected from the group consisting of HOXA7, CENPA, DNMT1, INHBA, BIRC5, CXCL8, IVL and CBX7.

26. The kit of claim 24, comprising a means for quantifying the expression or concentration of:
a) all of HOXA7, CENPA, NEK2, FOXM1, TOP2A, BIRC5, MMP13, CXCL8, NR3C1, IVL, CBX7 and S100A16; and
b) at least 1 of the biomarkers selected from the group consisting of DNMT1 and INHBA.

27. The kit of claim 24, comprising a means for quantifying the expression or concentration of HOXA7, CENPA, NEK2, INHBA, FOXM1, TOP2A, BIRC5, MMP13, CXCL8, NR3C1, IVL, CBX7 and S100A16.

28. The kit of claim 24, comprising a means for quantifying the expression or concentration of HOXA7, CENPA, NEK2, DNMT1, INHBA, FOXM1, TOP2A, BIRC5, MMP13, CXCL8, NR3C1, IVL, CBX7 and S100A16.

29. The kit of claim 24, wherein the means for quantifying the expression or concentration of the biomarkers is a microarray or one or more magnetic beads coated with oligonucleotides specific for the biomarkers whose expression or concentration is being quantified.

30. A method of treating cancer in a patient, comprising administering a cancer therapy to said patient, wherein the patient has been diagnosed as having cancer or is suspected of having cancer as determined by the method of claim 1.

31. A method of treating cancer in a patient, comprising testing for, screening for or diagnosing cancer according to claim 1 using a sample obtained from the patient, and administering a cancer therapy if the patient is diagnosed as having cancer, or is suspected as having cancer.


SEQUENCE LISTING (32 sequences)

SEQ ID NO: 1 - DNA, 20 bp, artificial sequence
  synthetic sequence; HOXA7 forward primer
gccaatttccgcatctaccc

SEQ ID NO: 2 - DNA, 20 bp, artificial sequence
  synthetic sequence; HOXA7 reverse primer
ggtagcggttgaagtggaac

SEQ ID NO: 3 - DNA, 20 bp, artificial sequence
  synthetic sequence; CENPA forward primer
ctgcacccagtgtttctgtc

SEQ ID NO: 4 - DNA, 20 bp, artificial sequence
  synthetic sequence; CENPA reverse primer
gagagtccccggtatcatcc

SEQ ID NO: 5 - DNA, 19 bp, artificial sequence
  synthetic sequence; NEK2 forward primer
cattggcacaggctcctac

SEQ ID NO: 6 - DNA, 23 bp, artificial sequence
  synthetic sequence; NEK2 reverse primer
gagccatagtcaagttctttcca

SEQ ID NO: 7 - DNA, 18 bp, artificial sequence
  synthetic sequence; DNMT1 forward primer
cgatgtggcgtctgtgag

SEQ ID NO: 8 - DNA, 21 bp, artificial sequence
  synthetic sequence; DNMT1 reverse primer
tgtccttgcaggctttacatt

SEQ ID NO: 9 - DNA, 21 bp, artificial sequence
  synthetic sequence; INHBA forward primer
gctcagacagctcttaccaca

SEQ ID NO: 10 - DNA, 23 bp, artificial sequence
  synthetic sequence; INHBA reverse primer
aaattctctttctggtccccact

SEQ ID NO: 11 - DNA, 21 bp, artificial sequence
  synthetic sequence; FOXM1 forward primer
actttaagcacattgccaagc

SEQ ID NO: 12 - DNA, 18 bp, artificial sequence
  synthetic sequence; FOXM1 reverse primer
cgtgcagggaaaggttgt

SEQ ID NO: 13 - DNA, 22 bp, artificial sequence
  synthetic sequence; TOP2A forward primer
cagtgaagaagacagcagcaaa

SEQ ID NO: 14 - DNA, 22 bp, artificial sequence
  synthetic sequence; TOP2A reverse primer
aagctggatcccttttagttcc

SEQ ID NO: 15 - DNA, 19 bp, artificial sequence
  synthetic sequence; BIRC5 forward primer
agaactggcccttcttgga

SEQ ID NO: 16 - DNA, 18 bp, artificial sequence
  synthetic sequence; BIRC5 reverse primer
acactgggccaagtctgg

SEQ ID NO: 17 - DNA, 20 bp, artificial sequence
  synthetic sequence; MMP13 forward primer
tgagctggactcattgtcgg

SEQ ID NO: 18 - DNA, 20 bp, artificial sequence
  synthetic sequence; MMP13 reverse primer
aggtagcgctctgcaaactg

SEQ ID NO: 19 - DNA, 22 bp, artificial sequence
  synthetic sequence; CXCL8 forward primer
aagtttttgaagagggctgaga

SEQ ID NO: 20 - DNA, 23 bp, artificial sequence
  synthetic sequence; CXCL8 reverse primer
tggcatcttcactgattcttgga

SEQ ID NO: 21 - DNA, 20 bp, artificial sequence
  synthetic sequence; NR3C1 forward primer
tccctggtcgaacagttttt

SEQ ID NO: 22 - DNA, 20 bp, artificial sequence
  synthetic sequence; NR3C1 reverse primer
gctggatggaggagagctta

SEQ ID NO: 23 - DNA, 20 bp, artificial sequence
  synthetic sequence; IVL forward primer
tgcctgagcaagaatgtgag

SEQ ID NO: 24 - DNA, 20 bp, artificial sequence
  synthetic sequence; IVL reverse primer
ttcctcatgctgttcccagt

SEQ ID NO: 25 - DNA, 21 bp, artificial sequence
  synthetic sequence; CBX7 forward primer
cgagtatctggtgaagtggaa

SEQ ID NO: 26 - DNA, 18 bp, artificial sequence
  synthetic sequence; CBX7 reverse primer
gggggtccaagatgtgct

SEQ ID NO: 27 - DNA, 21 bp, artificial sequence
  synthetic sequence; S100A1 forward primer
caagatcagcaagagcagctt

SEQ ID NO: 28 - DNA, 19 bp, artificial sequence
  synthetic sequence; S100A1 reverse primer
gagcttatccgcagccttc

SEQ ID NO: 29 - DNA, 22 bp, artificial sequence
  synthetic sequence; YAP1 forward primer
acaatgacgaccaatagctcag

SEQ ID NO: 30 - DNA, 23 bp, artificial sequence
  synthetic sequence; YAP1 reverse primer
ccactgtctgtactctcatctcg

SEQ ID NO: 31 - DNA, 20 bp, artificial sequence
  synthetic sequence; POLR2A forward primer
tccgtattcgcatcatgaac

SEQ ID NO: 32 - DNA, 20 bp, artificial sequence
  synthetic sequence; POLR2A reverse primer
tcatccatcttgtccaccac